(19) United States
(12) Patent Application Publication
(10) Pub. No.: US 2025/0146182 A1
(43) Pub. Date: May 8, 2025

(54) METHOD FOR DEGUMMING SILK FIBERS

(71) Applicant: YEDA RESEARCH AND DEVELOPMENT CO. LTD., Rehovot (IL)

(72) Inventors: Ulyana SHIMANOVICH, Rehovot (IL); Dror ELIAZ, Rehovot (IL)

(73) Assignee: YEDA RESEARCH AND DEVELOPMENT CO. LTD., Rehovot (IL)

(21) Appl. No.: 18/865,701

(22) PCT Filed: Jun. 15, 2023
(86) PCT No.: PCT/IL2023/050623

§ 371 Date: Nov. 14, 2024

(30) Foreign Application Priority Data
Jun. 16, 2022 (IL) 294040

(51) Int. Cl. D01C 3/02; C07K 14/435; D01F 4/02

(52) U.S. Cl. CPC D01C 3/02; C07K 14/43586; D01F 4/02; D10B 2401/063

(57) ABSTRACT

The present invention is directed towards degumming a silk fiber, obtaining high quality silk fibroin solutions and the reconstitution of silk. The invention further relates to a method of accurately and precisely determining mechanical properties of biological fibers such as silk fibers.

### FIELD OF THE INVENTION

This invention relates to a method for degumming silk fibers and for obtaining high quality silk fibroin solutions. The invention further provides silk fiber and a reconstituted silk soluble form. The invention further relates to a method of accurately and precisely determining mechanical properties of biological fibers such as silk fibers.

### BACKGROUND

Natural silk fibroin fibers represent a class of protein building blocks that can be further functionalized and/or re-processed into many different material formats. Standard methods of determining the exceptional mechanics of silk fibroin fibers involves removal of a gum coating layer, named sericin, via boiling the fibers in the aqueous solution containing Na₂CO₃ and further analyzing and calculating mechanics by applying a shape approximation of a perfect cylinder. Such approaches, however, are not ideal neither for preserving the protein (fibroin) components from thermal damage upon gum removal, nor for extrapolation of the correct silk fiber mechanics, whose shape is not a perfect cylinder, but is rather asymmetrical with multiple defects—an abundant phenomenon in biological fibers.

### SUMMARY

In one embodiment, the present invention relates to a formulation developed for a non-thermal sparing removal of the sericin gum layer, which preserves fibroin protein integrity, secondary structure and consequently improving fiber mechanics and thermal stability of the protein material. This method further enables obtaining soluble silk of high purity and rheological characteristics comparable to those of native silk (i.e., silk extracted directly from the silk gland via dissection) when the fibers are further re-solubilized.

Furthermore and in one embodiment, the present invention establishes a novel approach for the determination of mechanical characteristics of silk fibers, the method accounts for the fiber asymmetry and internal/external defects. The developed approaches are beneficial not only for the generation of silk-based materials with tailored and enhanced properties, but also for correctly establishing the mechanical characteristics of asymmetrical fibrous materials made of natural and synthetic building blocks. The present invention establishes a new approach for the determination of “true” mechanical characteristics of biological fibers like silk, which accounts for the fibers' asymmetry and the presence of defects.

In one embodiment this invention provides a method for removing at least part of a gum layer from silk fibers, said method comprising: incubating silk fibers in an incubating medium, the incubating medium comprising a hydroxide. In one embodiment this invention provides a method for removing at least part of a gum layer from silk fibers, said method comprising: incubating silk fibers in an incubating medium, the incubating medium comprising a hydroxide; wherein the method is carried out at a temperature ranging between 5 to 60 degrees Celsius. In one embodiment the incubating medium consists of one hydroxide and water. In one embodiment the incubation of the disclosed method consists of a single incubating step.

In one embodiment the method is carried out at a temperature ranging between 5 to 60 degrees Celsius. In one embodiment the hydroxide is selected from: NaOH, LiGH, KOH, RbOH, CsOH, Ca(OH)₂, Sr(OH)₂ or Ba(OH)₂ or a combination thereof.

In one embodiment the concentration of the hydroxide in the incubating medium ranges from between about 0.1M to 1M. In one embodiment the incubating medium further comprises at least one buffer. In one embodiment the incubating medium further comprises at least one salt. In one embodiment the incubating is carried out from between 1 minute to 60 minutes. In one embodiment the method further comprises rinsing the silk fibers in a liquid after the incubation. In one embodiment the liquid comprises water or a solution. In one embodiment the method further comprises drying the silk fibers after rinsing in the liquid. In one embodiment the method further comprises separating the silk fibers into individual fibers.

In one embodiment this invention provides a degummed silk fiber comprising a core and a gum layer wherein the gum layer comprises 0% to 20% of the cross-sectional area of the degummed silk fiber.

In one embodiment the degummed silk fiber exhibits a Young's modulus of at least about 10 GPa. In one embodiment the degummed silk fiber exhibits a Young's modulus which is at least 1.5 greater than for an untreated silk fiber. In one embodiment the degummed silk fiber exhibits a tensile strength of at least about 400 MPa. In one embodiment the degummed silk fiber exhibits a tensile strength of at least 1.25 times greater than for an untreated silk fiber. In one embodiment the degummed silk fiber exhibits a strain at break of at least about 0.2. In one embodiment the degummed silk fiber exhibits a Weibull characteristic strength parameter (a) of at least about 400 MPa.

In one embodiment the degummed silk fiber exhibits a Weibull characteristic strength parameter (a) of at least 1.2 times greater than for an untreated silk fiber. In one embodiment the degummed silk fiber exhibits a Weibull shape parameter (P) of at least about 4. In one embodiment the degummed silk fiber is produced by a method as described hereinabove.

In one embodiment, this invention provides a reconstituted silk fibroin material, the material comprising silk fibroin proteins sourced from a degummed silk fiber as described hereinabove and a liquid. In one embodiment the liquid comprises water. In one embodiment the liquid comprises a buffer.

In one embodiment, this invention provides a method for determining average mechanical properties of a plurality of asymmetric fibers, the method comprising:
a) using a confocal microscope to obtain a plurality of cross-sectional images along the length of one of the asymmetric fibers;
b) calculating cross-sectional area of the asymmetric fiber;
c) calculating an average cross-sectional area along the length of the asymmetric fiber;
d) measuring the extension versus load for the asymmetric fiber until the asymmetric fiber breaks;
e) using the average cross-sectional area to obtain a stress-strain plot for the asymmetric fiber;
f) obtaining maximum strength and Young's modulus from the stress-strain plot for the asymmetric fiber;
g) performing stages ‘a’ to ‘f’ for a plurality of asymmetric fibers to obtain a plurality of stress/strain data sets; and
h) calculating an average maximum strength and average Young's modulus for the plurality of asymmetric fibers.

In one embodiment the asymmetric fiber is stained with a dye prior to step (a). In one embodiment the asymmetric fiber is of biological origin. In one embodiment the asymmetric fiber is degummed. In one embodiment the asymmetric fiber is coated with an additional material.

In one embodiment, the method further comprises utilizing a two-parameter Weibull distribution, comprising;
a) ranking the maximum strength for the plurality of stress/strain data sets from lowest to highest and assigning the data set a number;
b) calculate the natural log of the maximum strength for each data set;
c) calculate the probability of failure, f(σ), for each data set;
d) plot the natural log of the maximum strength versus ln(−ln(1−f(σ)); and
e) extract the shape parameter, β, from the gradient and the scale parameter, α, from α=exp(intercept/β) from the plot in ‘d’.

In one embodiment the asymmetric fiber is stained with a dye prior to step (a). In one embodiment the asymmetric fiber is of biological origin. In one embodiment the asymmetric fiber is degummed. In one embodiment the asymmetric fiber is coated with an additional material.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A to 1D show a representation of microscopy analysis of degummed silk fibers obtained using different degumming approaches.

FIG. 1E to FIG. 1I show microscopy analyses of degummed RSF fibers obtained by using different degumming approaches. Confocal images of the untreated (FIG. 1E) and degummed fibers (FIG. 1F: Na₂CO₃, FIG. 1G: NaOH 0.1M, FIG. 1H: NaOH 0.5M and FIG. 1I: NaOH 1M). The fibers stained with Nile Red dye, where the left side shows longitudinal 3D confocal image, and the right side shows the longitudinal 3D confocal processed image.

FIG. 2 shows an analysis of the mechanical behavior of as-produced silk and degummed fibroin protein fibers. FIG. 2A represents the cross-section distribution. FIG. 2B represents the true stress-strain curves of the untreated and degummed silk fibers and graphical representation of ‘true’ mechanical properties are show in FIG. 2C for the true strain, FIG. 2D for the true Young's modulus, FIG. 2E for the true tensile strength and FIG. 2F shows a graphical representation of the calculated two-parameter Weibull distribution plots of true values for the strength of different fibers.

FIG. 3 is a representation of thermogravimetric analysis (TGA) for the fibers before and after different degumming treatments.

FIG. 4 represents the structural characteristics and the mechanism of silk fibers' degumming.

FIG. 5 represents a schematic of different degumming treatments and processes.

FIG. 6 illustrates cooperative analysis for the measured cross-sectional area of untreated fibers, fibers degummed via standard approach and fibroin fibers treated with NaOH-based solution.

FIG. 7 depicts SEM images of NaOH 1M treatment and decomposition of the microscale fiber into the separated nanofibrils.

FIG. 8 represents stress-strain curves of untreated silk-fiber (FIG. 8A), Na₂CO₃ treated silk-fiber (FIG. 8B), NaOH 0.1M treated silk-fiber (FIG. 8C), NaOH 0.5M treated silk-fiber (FIG. 8D) and NaOH 1M treated silk-fiber (FIG. 8E).

FIG. 9 is a representation of thermogravimetric analysis (TGA) for the fibers before and after different degumming treatments: FIG. 9A is for untreated silk-fibers. FIG. 9B is for NaOH 0.1M. FIG. 9C is for NaOH 1M.

FIG. 10 shows TGA analysis for the fibers before and after different degumming treatments. There are 5 repeats of TGA scans for degummed silk fibroin fibers, showing the onset of TGA analysis for degummed fibers with NaOH. FIG. 10A shows dynamic mechanical analysis (DMA) of degummed fibers; the storage moduli are represented by the continuous lines and the loss moduli by dashed line.

FIG. 10B shows the glass transition temperature of degummed fibers.

FIG. 11 shows properties relating to the structural characteristics and the mechanism of soluble silk fibroin. FIG. 11A shows small-angle X-ray scattering (SAXS) analysis of soluble silk fibroin; azimuthally-interacted background-subtracted solution X-ray scattering absolute intensity, I, as a function of the magnitude of the scattering vector, q, from native silk and soluble RSF obtained from the different degummed approaches. FIGS. 11B and 11C show rheology analysis of changes in viscosity in response to applied shear, with increasing shear rate from 0.1 s⁻¹ to 500 s⁻¹ and back from 500 s⁻¹ to 0.1 s⁻¹. The following samples were analyzed: native silk fibroin solution obtained from the silkworm gland via dissection, RSF solution obtained via chemical resolubilization of the silk fibers degummed in the presence of Na₂CO₃, NaOH 0.1M, and NaOH 0.5M and NaOH 1M. The shade's colors represent the standard deviation (mean±sd, n≥3).

FIG. 12 shows an algorithm description of the process of the cross-section analysis that is done by a Matlab script. First (first step), two channels of 3D confocal images are taken then (second step), the image is post-process by the Imaris program. At the third step, the script converts the two channels' image into binary images and then collect the cross-section area for each pixel along the silk fiber (axes).

FIG. 13 shows a statistical analysis for the mechanical measurements. One-way analysis of variance (ANOVA) was conducted between all the samples in order to determine whether significant differences existed among the mean values. The difference among groups was statistically significant at p<0.05.

FIG. 14 shows Fourier Transform Infrared spectroscopy (FTIR) analyses of silk fibers, degummed and untreated. FTIR spectra of silk fibers untreated (FIG. 14A), Na₂CO₃ (FIG. 14B), NaOH 0.1M (FIG. 14C), NaOH 0.5M (FIG. 14D), and NaOH 1M (FIG. 14E). FIG. 14F shows a bar chart of the relative amounts of the β-sheets at 1610-1635 cm⁻¹, the anti-parallel β-sheets at 1690-1705 cm⁻¹, random coil and α-helixes at 1635-1665 cm⁻¹, and β-turns at 1665-1690 cm⁻¹.

### DETAILED DESCRIPTION

Natural silk cocoon fibroin protein of silkworm, Bombyx mori (B.mori), is widely utilized in a variety of biomedical applications, in addition to the traditional textile industry. The FDA-approved B.mori fibroin is used as a versatile biomaterial in the form of films, membranes, gels, sponges, powders, scaffolds, and nanoparticles. Such popularity is gained due to its exceptional mechanical performance, controllable porosity, oxygen and water permeability, bio-degradability, hemostatic properties, non-cytotoxicity, low antigenicity, and non-inflammatory characteristics. In order to take advantage of these exceptional characteristics, pre-treatment in the form of separation between the fibroin protein core component and sericin gum coating layer is required. The fraction of the sericin layer varies between 25-30% of the total fiber weight. The fibroin core fiber is characterized by a highly hierarchical structural organization. The fibroin core fiber, which is 70-75 wt % of the silk fiber is made of two microscale fibers of ˜10 μm each in diameter, where the single fibroin fiber is composed of aligned bundle of fibroin nanofibrils. The nanofibrils are made of self-assembled fibroin protein. The fibroin itself is a large globular protein of ˜400-450 kDa in size, which contains two subunits of heavy and light chains, that are linked via a single disulfide bond. The structural organization of proteins inside the single nanofibril alternates between the crystalline and disordered regions, where the fraction of the crystalline regions defines the strength of the final fiber. The overall mechanics of silk fibers is evolved through structural transformation of soluble fibroin protein, stored inside the gland (silk feedstock), from a relatively disordered state (random coil conformation) into highly ordered β-sheet rich solid fiber via a spinning process.

Traditionally, sericin layers are removed by boiling silk fibers in aqueous solutions containing Na₂CO₃ for about one hour. Such procedures often damage fibroin protein components either by changing its fold or by separating between the heavy and the light chain protein subunits. Such damage reduces the mechanical performance of the fibroin fibers. Further, it affects their thermal stability and further reduces the quality of the reconstituted silk fibroin (RSF) obtained via chemical re-solubilization.

In one embodiment, this invention provides a process for gentle removal of the gum layer from a silk fiber without imposing thermal damage. The inventive process preserves the protein secondary structure and its crystallinity. In addition, the process improves mechanical performance and thermal stability of the fibers. Furthermore, and in one embodiment, when fibroin fibers treated by a process of this invention are chemically resolubilized to obtain aqueous solution of RSF, the rheological properties of the resulting RSF are comparable to those of native silk from the silkworm gland.

To improve the mechanical properties of fibroin fibers, the present disclosure shows a formulation that efficiently removes the sericin gum layer, at room temperature, and preserves structural hierarchy of silk material, including the molecular structure of the fibroin protein, the nanostructure of the fibroin nanofibrils and thus, the integrity of the final fibroin microfiber. A precise analysis of the mechanical characteristics was enabled via determination of the actual initial cross-sectional area of the asymmetrical silk fibers. As shown herein, removing the sericin layer with developed formulation improves the Young's modulus (by 96% and 23%) and strength (by 52% and 47%) of the fiber, without affecting its strain, compared to untreated and to boiled fibers, respectively. Furthermore, the treatment yields to a lower variance in the defects present along the fibers. The developed approaches are beneficial not only for the generation of silk-based materials with tailored properties, but also for establishment of mechanical characteristics of asymmetrical fibrous biological materials made of natural building blocks.

In the presently disclosed invention, a simple method for the gentle removal of the sericin layer without imposing thermal damage to the protein fiber core, grounded on sodium hydroxide (NaOH) is provided. NaOH is used in many protein extraction protocols to dissolve impurities, minerals, pigments, and specific proteins. In order to break down interprotein non-covalent interactions and remove the sericin layer by NaOH, conditions such as temperature, NaOH concentrations and time exposure to NaOH are considered. The present disclosure of silk fibers' treatment preserves the secondary structure and crystallinity of the fibroin protein component, protects the fiber's morphology, and improves the mechanics and the stability of the fibers compared to untreated silkworm fibers and to boiled fibers boiled in a standard Na₂CO₃-containing solution. When fibroin fibers are further chemically re-solubilized, in order to obtain aqueous protein feedstock, a reconstituted silk fibroin (RSF), the rheological properties of the RSF are comparable to those of native silk extracted from the silkworm gland. Notably, since aqueous silk is defined as a flow-sensitive material, rheological characteristics of silk play an important role in silk fiber production via spinning. Thus, “improving” the rheological characteristics of RSF improves the generation of artificial silk fibers, gels, or films with tailored mechanics properties. Additionally, a new methodology for the determination of the actual initial cross-sectional area of the silk fibroin fibers is shown herein. The correct determination of the cross-sectional area usually serves as a base for calculations of the strength of biological and non-biological fibers. The approach disclosed herein includes a staining assay enabling the differentiation between the two main components of silk fibers, namely fibroin protein and sericin gum. The method facilitates extraction of the volume and cross-sectional area parameters of the non-uniform fibers from confocal microscopy analysis, which were further used for the comprehensive analysis of the mechanical properties of the fibers. This procedure considers the asymmetrical shape of the fibers. The two-parameter Weibull distribution enabled analyzing presence of defects along the fiber surface morphology, resulting in a proper understanding of the mechanical capabilities of fibroin fibers.

Silk fibroin fibers represent a class of protein building blocks that can be further functionalized and/or re-processed into different material formats. Standard methods of determining exceptional mechanics of these fibers involve removal of the sericin gum coating layer via boiling fibers in the presence of Na₂CO₃, and further analyzing and calculating mechanics via applying shape approximation of the perfect cylinder. Such approaches, however, are not ideal neither for preserving protein (fibroin) component from the thermal damage upon gum removal, nor for extrapolation of the correct and “true” silk fibers' mechanics, whose natural shape is asymmetrical with multiple defects—an abundant phenomenon in biological fibers. Disclosed herein is a formulation for non-thermal sparing removal of sericin gum layer, which preserves fibroin protein integrity, its secondary structure, and consequently improving fiber mechanics and fibers stability. The method has been demonstrated and enables obtaining soluble silk protein of high purity and with rheological characteristics similar to those of native silk feedstock originally stored in an animal's silk gland. Secondly, a new approach for determination of correct mechanical (or ‘true’) characteristics of silk fibers, which accounts for a fibers' asymmetry and the presence of multiple defects. The developed approaches are beneficial not only for the generation of silk-based materials with tailored properties, but also for establishment of mechanical characteristics of asymmetrical fibrous biological materials made of natural and synthetic building blocks.

As referred to herein “true”, in reference to silk fibers and their associated properties, refers to their characteristics as accounted for by virtue of their natural asymmetric shape and may contain defects. In some embodiments a silk fiber comprises at least one defect. In some embodiments the silk fiber structure is asymmetric. The term “true” therefore accounts for the fact that silk fibers are non-idealized structures, for example a uniform tube, but rather comprise various structural elements. Such structural elements can include defects, multi-layered structures and asymmetries, or any other internal structure that is not uniform. Being able to determine the total cross sectional area, and thus volume, of silk fibers allows for the calculation of true mechanical properties.

In some embodiments the gum layer in silk comprises sericin. In some embodiments the gum layer comprises a protein-based structure. In some embodiments the gum layer is referred to as the “outer layer” of the silk fiber. In some embodiments the gum layer is referred to as the “coat” or “coating” layer or as the “shell” of the silk fiber. In some embodiments the “outer layer” comprises any material that is not fibroin-based. In some embodiments the outer layer comprises glycoproteins. In some embodiments the outer layer comprises lipids. In some embodiments, the gum layer surrounds the fibroin inner fiber. In one embodiment, the gum layer surrounds a portion of the fibroin fiber. In one embodiment, the gum layer surrounds at least a portion of two fibroin fibers.

In one embodiment, this invention provides a novel method for evaluating silk mechanics. Currently, fiber mechanics calculations rely on a perfect cylinder shape approximation of the fiber. However, the cross-section of silk fibers exhibits a non-uniform and non-cylindrical shape with the presence of multiple defects. Inaccuracy in shape approximation dramatically changes the tensile strength value as well as fiber strength. In one embodiment, methods of this invention provide an accurate and precise procedure for determining fiber mechanics. According to this aspect and in one embodiment, methods of this invention utilize an accurate cross section of the fiber for the measurements of e.g. tensile strength. In one embodiment, in order to find an accurate cross section of a fiber, staining assays are used. In one embodiment, staining assays enable differentiation between components of silk fibers, for example between fibroin and sericin. In one embodiment, a microscope is used to evaluate the cross section of a stained fiber. For an asymmetric fiber, confocal microscopy facilitates obtaining cross-sectional measurements along a fiber. An average cross section is thus obtained for a fiber in which the cross section varies along the length of the fiber. Accordingly and in one embodiment, a script is used to extract the volume and cross-section parameters of the non-uniform fibers from confocal microscopy analysis. In embodiments of this invention, the volume and cross-section parameters were further used for a comprehensive analysis of the silk fiber mechanics.

One goal of the present invention is to establish a more accurate and precise approach for determining mechanical properties of silk fibers. In one embodiment, processes of this invention include non-damageable removal of gum component. Processes of this invention further comprise staining procedures to enable differentiation between biopolymeric composite components in some embodiments. In one embodiment, an analytical process is used for determination of the mechanical properties of non-uniform fibers.

### Development of Formulation for Silk Fibers Degumming:

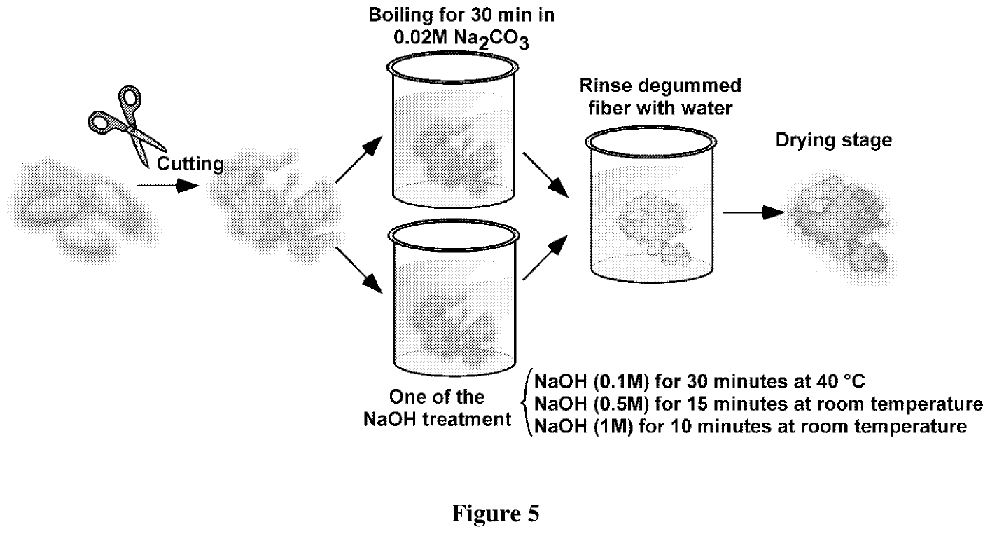

In one embodiment, this invention provides a fiber degumming method which eliminates the thermal damage imposed by a standard protocol of silk fibers degumming. The standard protocol currently used, involves boiling of silk fiber at 100° C. in aqueous sodium bicarbonate (Na₂CO₃, 0.02M, 30 min to 1 hour). In one embodiment, this invention provides a detailed screening of media for a delicate sericin gum removal (see Table 1 for summary of liquid solutions). Short incubation of silk fibers in the presence of NaOH of different molarity values ranging from about 0.1 to 1M, at room temperature, results in high yields of sericin removal (see FIG. 5 and Table 1). The sericin is originally ˜30 wt % of the initial fiber weight. In one embodiment, all the sericin layer was removed by a process of this invention. In one embodiment, the sericin layer which is ˜30% weight of the silk fiber has been removed completely by a process of the invention as described herein. In one embodiment, more than 75% of the sericin layer (which is ˜30% weight of the original silk fiber) has been removed by a process of the invention as described herein. In one embodiment, at least 90%, or at least 95%, or at least 99%, or at least 99.9% of the sericin layer weight has been removed by processes of this invention. In one embodiment, at least 90%, or at least 95%, or at least 99%, or at least 99.9% of the sericin layer volume has been removed by processes of this invention.

In one embodiment, the term “a” or “one” or “an” refers to at least one. In one embodiment the phrase “two or more” may be of any denomination, which will suit a particular purpose. In one embodiment, “about” or “approximately” may comprise a deviance from the indicated term of ±1%, or in some embodiments, −1%, or in some embodiments, ±2.5%, or in some embodiments, ±5%, or in some embodiments, ±7.5%, or in some embodiments, ±10%, or in some embodiments, ±15%, or in some embodiments, ±20%, or in some embodiments, ±25%.

As used herein “room temperature” refers to a temperature range of about 10 to about 25 degrees Celsius. As used herein “room temperature” refers to a temperature range of about 5 to about 50 degrees Celsius. In some embodiments “room temperature” is about 23 degrees Celsius. In some embodiments, the incubation of silk is carried out at a temperature ranging between 20 to 40 degrees Celsius. In some embodiments the incubation of silk is carried out at between 40 to 60 degrees Celsius. In some embodiments, the incubation of the silk is carried out at about 40 degrees Celsius. In some embodiments the incubation of silk is carried out at between 5 to 60 degrees Celsius.

In some embodiments, fibers of this invention comprise a biological fiber. In some embodiments the fiber consists of a biological fiber. In some embodiments, fibers of this invention comprise synthetic fibers.

In some embodiments, the time period used for incubation of silk fibers is between 0 to 5 mins. In some embodiments incubation of silk fibers is between 0 to 10 mins. In some embodiments incubation of silk fibers is between 0 to 20 mins. In some embodiments incubation of silk fibers is between 0 to 30 mins. In some embodiments incubation of silk fibers is between 0 to 60 mins. In some embodiments incubation of silk fibers is between 10 to 20 mins. In some embodiments incubation of silk fibers is between 20 to 30 mins. In some embodiments incubation of silk fibers is between 30 to 60 mins. In some embodiments incubation of silk fibers is up to 1 hour. In some embodiments incubation of silk fibers is over 1 hour. In one embodiment, incubation time is between 0.1 min and 60 min or between 0.1 min and 120 min. In some embodiments the incubation of silk fibers ranges between 1 to 3 hours.

FIG. 5 is a schematic depiction of different degumming treatments and processes. The illustration depicts B. mori cocoons which are cut into small pieces. Subsequently, four different degumming treatments are represented to remove the sericin layer. Treatments include: Na₂CO₃ and NaOH (0.1M, 0.5M, and 1M). After degumming, the fibers are extensively washed and/or rinsed with water and dried and room temperature. In one embodiment, after the incubation stage the degummed fibers are washed/rinsed in water. In one embodiment, after the incubation stage the degummed fibers are washed/rinsed in a solution. In some embodiments the terms “rinsing” and “washing” are used interchangeably. In some embodiments rinsing/washing can comprise washing the degummed fibers under a stream of water or otherwise placing the degummed fibers in a receptacle of water and/or solution e.g., a beaker. In some embodiments rinsing comprises dialysis. Dialysis can be carried out using any solution composition, optimized for the desired pH and ionic strength, for example: water, buffer solution or a mixture containing number of solvents of aqueous or organic nature. The membrane, filter parameters, washing time and frequency, solution volume and concentration are selected to optimize for a particular analyte. In some embodiments, rinsing/washing or degummed fibers is carried out more than once. In some embodiments, the water comprises deionized, purified and/or filtered water.

After the degummed silk fibers are rinsed/washed in water/solution, the degummed fibers can be obtained by drying. In some embodiments, drying can be carried out under any conditions that preserve the structural integrity of the degummed fibers. In some embodiments, the degummed fibers are dried under a flow of a gas. In one embodiment, the gas is an inert gas, e.g., nitrogen or argon. In one embodiment, the degummed silk fibers are dried in the presence of a vacuum, in a vacuum chamber and/or in a dry box. In other embodiments the degummed silk fibers are left to dry under ambient conditions. In some embodiments, the method depicted in FIG. 5 produces individual fibers, mats of fibers, bundles of fibers and/or a plurality of fibers. In some embodiments, following drying, fibers are separated to obtain whichever desirable form. In some embodiments, one or more fibers are selected from a formed bundle of dried, degummed fibers. In one embodiment, a single fiber is withdrawn from a bundle of dried, degummed fibers for subsequent treatment/analysis. In one embodiment, single fibers are individually withdrawn from a bundle of dried, degummed fibers for subsequent treatment/analysis. In one embodiment, single fibers are individually used for further testing, and the results of the tests of the single fibers is further used for analysis as described herein.

TABLE 1

| | Cocoon | Concentration | Incubation | Temperature | Dry weight | Dry wet weight | Sericine |
|---|---|---|---|---|---|---|---|
| Sample | weight (mg) | (M) | (Min) | (° C.) | (mg) | ratio | removal % |
| Na₂CO₃ | 500 | 0.02 | 30 | 100 | 362.55 | 0.7251 | 27.49 |
| NaOH | 500 | 1 | 10 | RT | 364.1 | 0.7282 | 27.18 |
| NaOH | 500 | 0.5 | 15 | RT | 356.1 | 0.7122 | 28.78 |
| NaOH | 500 | 0.1 | 30 | 40 | 369.9 | 0.7398 | 26.02 |

In some embodiments, silk fibers are incubated in the presence of hydroxide (e.g. sodium hydroxide (NaOH)). As referred to herein and in one embodiment, an “incubating solution”, “incubation solution” ˜, “incubation medium”, “incubating medium”, “medium” or “solution” refers to a liquid which comprises at least one substance, in which the fibers are placed. Other equivalent terms for the incubation medium are also used, as known to experts in the art. In one embodiment, silk fibers are incubated in solutions comprising any of the following: NaOH, LiOH, KOH, RbOH, CsOH, Ca(OH)₂, SR(OH)₂, BA(OH)₂ or any combination thereof. In some embodiments, silk fibers are incubated in a solution comprising one hydroxide. In some embodiments, silk fibers are incubated in a solution comprising one hydroxide and water. In some embodiments, silk fibers are incubated in a solution consisting of one hydroxide and water. In some embodiments, silk fibers are incubated in a solution comprising at least one hydroxide and water.

In some embodiments, the incubating solution further comprise at least one buffer. In one embodiment, the incubating solution further comprise at least one weak acid, weak alkali and/or salts. In other embodiments, the incubating solution further comprises at least one strong acid and/or strong alkali.

In some embodiments, 100% of the sericin layer is removed by processes of this invention. In some embodiments, about 100% of the sericin layer is removed by processes of this invention. In some embodiments, the incubation process partly removes the gum layer. In some embodiments 0% to 50% of the gum layer is removed by a method of the present invention. In some embodiments, 0.001% to 50% of the gum layer is removed by methods of the present invention. In other embodiments 50% to 100% of the gum layer is removed by a method of the present invention. In some embodiments, as defined herein “partly” or “in part” refers to partial removal of a gum layer, which comprises any amount that is not 100%. In one embodiment, the whole gum layer is removed by methods of this invention. In one embodiment, 100% of the gum layer is removed by methods of this invention.

In some embodiments, the sericin or gum layer comprises 0% to 20% of the cross-sectional area and/or volume of a degummed silk fiber. In some embodiments the sericin or gum layer comprises 0% to 10% of the cross-sectional area and/or volume of a degummed silk fiber. In some embodiments the sericin or gum layer comprises 0% to 5% of the cross-sectional area and/or volume of a degummed silk fiber. In some embodiments the sericin or gum layer comprises 0% to 20% of the material mass of a degummed silk fiber. In some embodiments the sericin or gum layer comprises 0% to 10% of the material mass of a degummed silk fiber. In some embodiments the sericin or gum layer comprises 0% to 5% of the material mass of a degummed silk fiber. In some embodiments the sericin or gum layer comprises 0% of the material mass of a degummed silk fiber. In some embodiments, the sericin or gum layer comprises less than 0.1%, or less than 0.01%, or less than 0.001% of the material mass of a degummed silk fiber.

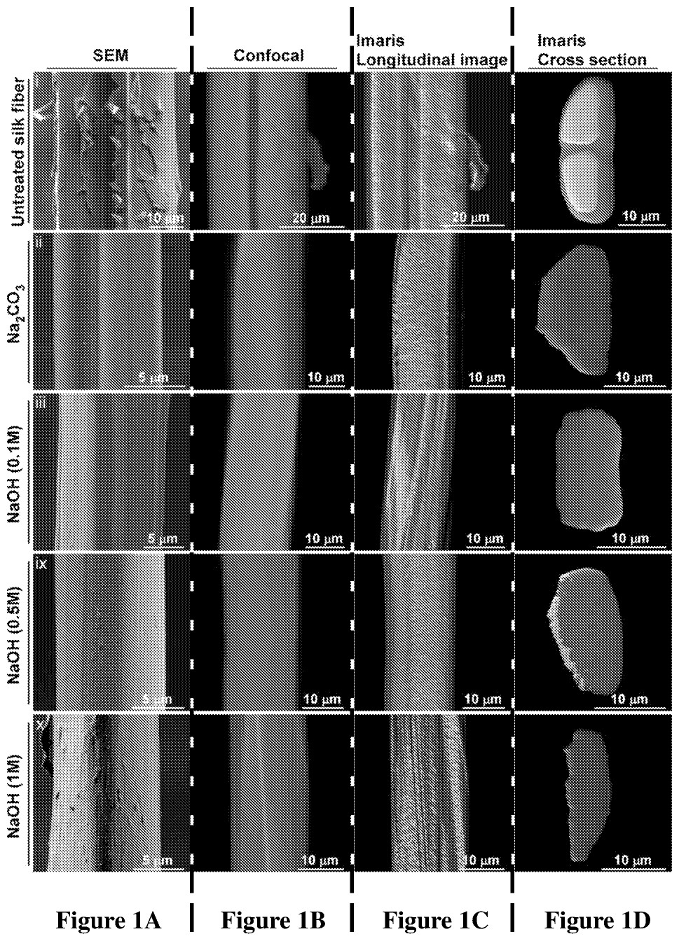

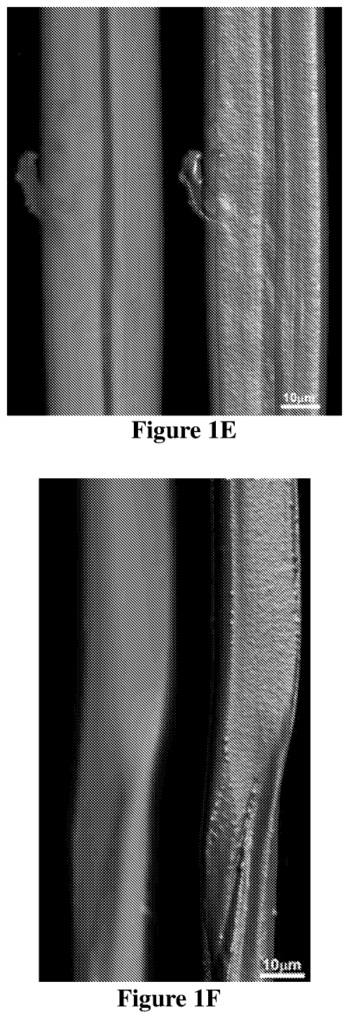

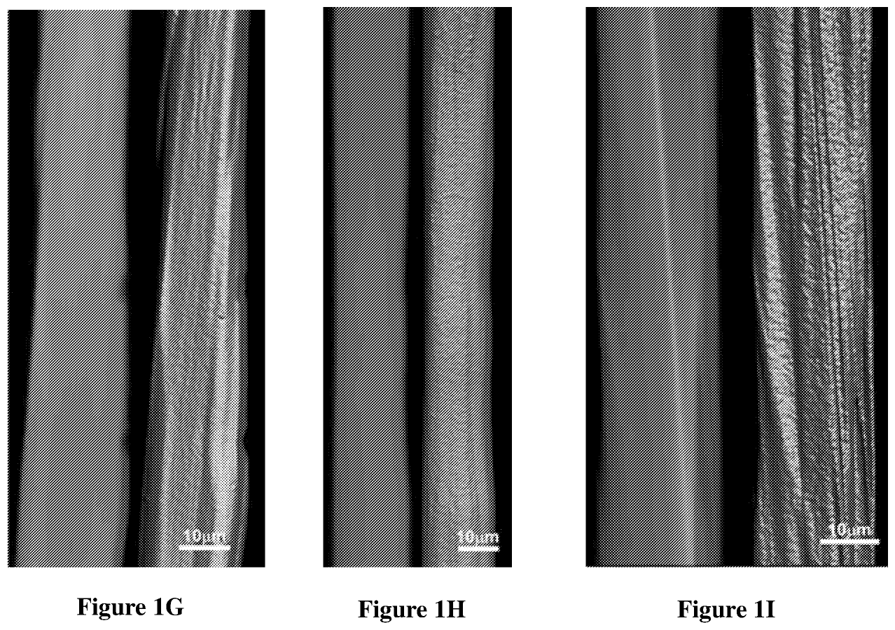

A detailed morphological analysis was performed using scanning electron microscopy (SEM) as shown in FIG. 1A. The analysis revealed the presence of a remaining thin sericin layer when the removal was done by the standard protocol using Na₂CO₃. In contrast, for all NaOH treatments, the gum component was absent (FIG. 1A-FIG. 1D). In addition to visual observations of the efficiency of the sericin removal, the asymmetry in fibroin fibers shape can be seen. As noted herein above, such asymmetry represents an obstacle in correct determination of the mechanical characteristics of fibroin fibers. This is because existing methods of data analysis obtained from mechanical measurements typically rely on an approximation using a perfect cylindrical shape of the fiber, lacking any defects. Thus, such assumptions cannot be applied to biological fibers, including silk fibroin fibers. To address this challenge, the presently disclosed subject matter details a method that enables:
1. distinguishing between the sericin coating layer and the fibroin protein core fiber;
2. measuring the cross-sectional area of asymmetrical composite fibers as well as of each component separately; and
3. establishing a fiber volume with high precision.

As found by the present invention, these measured parameters are essential for further calculation of accurate mechanical properties of asymmetrical biological fibers.

As used herein the term “asymmetry” refers to any structure that is not perfectly symmetrical. In some embodiments, “asymmetry” refers to any structure that is not uniform in part, or fully. In some embodiments “asymmetry” refers to a structure that comprises defects. In some embodiments the term “asymmetry” can be used interchangeably with the terms “non-uniform”, “ununiform”, “uneven” and/or “irregular” without limiting the scope of the invention.

In one embodiment the starting length and final length of a silk fiber, which undergoes degumming, is the same. Namely, there is no fragmentation of the initial fiber into smaller parts. In some embodiments there is partial fragmentation of the original silk fibers which undergo degumming. In other embodiments, the starting length and final length of a silk fiber, which undergoes degumming, is different i.e., it undergoes fragmentation during the process of degumming. Ideally, the integrity of the fiber remains after undergoing degumming processes, as disclosed in the various examples herein. In some embodiments the process is carried out in a solution without comprising an alcohol such as ethanol. In some embodiments the process is carried out in a solution without comprising urea.

In some embodiments the incubation solution comprises water and sodium hydroxide. In some embodiments the incubation solution consists of water and sodium hydroxide.

In some embodiments the efficiency of creating degummed fibers is between 50-100%. In some embodiments the efficiency of creating degummed fibers is between 60-80%. In some embodiments the efficiency of creating degummed fibers is between 80-100%. In some embodiments the efficiency of creating degummed fibers is between 90-100%. In some embodiments the efficiency of creating degummed fibers is between 95-100%. The efficiency, in this regard, refers to the percentage amount of fibers that are successfully degummed e.g., a percentage of the fibers that are fully degummed. Or otherwise, the efficiency refers to the percentage of individual fibers that are degummed e.g., all the degummed fibers are degummed by a certain percentage. In some embodiments, the efficiency of degumming can refer to both.

In some embodiments, the process described herein requires one incubation step to perform degumming. Otherwise referred to herein as a “single” incubation step. The single incubation step refers to placing the silk fibers in an incubation solution only once, for the purposes of degumming. This degumming, as disclosed elsewhere herein, can refer to a complete degumming or a partial degumming. Following the single step incubation step, other processes are carried out to obtain the final product.

### Cross-Sectional and Volumetric Analysis of Fibroin Fibers

FIG. 1A depicts scanning electron microscopy images of silk fibers. FIG. 1B depicts confocal microscopy imaging of the fibers. FIG. 1C depicts Imaris longitudinal imaging of the fibers. FIG. 1D depicts Imaris cross-section of the fibers.

In one embodiment, in order to measure the cross-section of the fiber and to calculate fiber volume, a staining procedure was combined with confocal microscopy analysis and image processing techniques (FIGS. 1C-1D). Intrinsic fluorescence signals in silk (measured excitation at 346 nm and emission at 434 nm) are utilized for measuring the cross-sectional area and the volume of asymmetric fibers containing both fibroin and the sericin components. In one embodiment, Nile red dye, an environmentally sensitive dye, which changes its fluorescence in response to changes in the extinction coefficient of the material, is used for selective staining of the fibroin components only. The Nile red dye emission peak is at about 635 nm.

As shown in FIG. 1D, for the untreated fiber and for the Na₂CO₃ treated fiber, the combination of intrinsic emission from the two components of the fiber with the emission of the Nile red dye from the fibroin component only, yields a precise optical distinction between the two components of the fiber (fibroin and sericin). Such distinction facilitates calculating the cross-section of the fiber and of any component thereof. As shown herein, such an accurate cross section assessment is highly significant for an accurate evaluation of the mechanical properties of the fiber.

In some embodiments, other suitable fluorescent dyes can be used for selective staining of various components within a fiber.

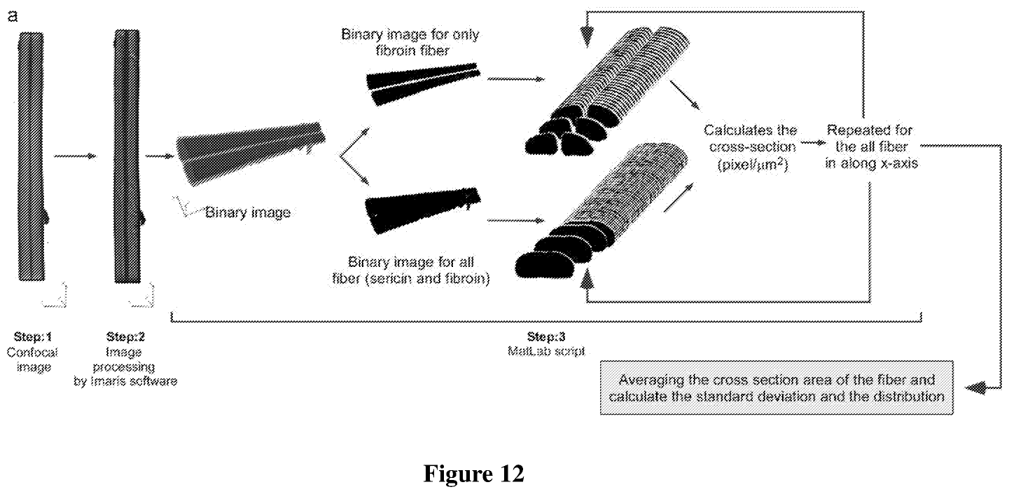

In some embodiments, z-stack confocal images are collected for each fiber. These confocal images are further processed into a 3D representation via image reconstruction by “Imaris” software (FIG. 1C and FIG. 1D). “Z-stack” refers to stacks of two-dimensional images, e.g., cross-sections, in any direction to complete a three-dimensional image. Typically, a z-stack will comprise 2D cross-sections along the length of a fiber. A schematic of z-stacking processes is depicted in FIG. 12.

In one embodiment, defining the boundaries between the fibroin and the sericin components, was enabled based on the differences between the blue and the red fluorescence signals as shown in FIG. 1D and as described herein above.

Fibroin and sericin components are two of a multitude of fiber components that can be utilized in embodiments of the present invention. In some embodiments, other fiber components can be distinguished using the methods of the present invention. In some embodiments, methods of the present invention can distinguish between different forms and structures of fibroin and sericin components themselves. In some embodiments the fibers comprise a biological material. In some embodiments the fibers comprise non-biological material. In some embodiments the fibers comprise organic material. In some embodiments the fibers comprise inorganic material. In one embodiment, the fiber is a natural fiber. In one embodiment, the fiber is a synthetic fiber. In one embodiment, the fiber comprises one component. In one embodiment, the fiber comprises two components. In one embodiment the fiber comprises three or more components. In one embodiment, each component of the fiber can comprise one part. In one embodiment, each component of the fiber can comprise more than one part. For example and in one embodiment, the fibroin components of an untreated silk fiber comprise two adjacent fibers as shown for example in FIG. 1D. All such parts and their specific cross section can be evaluated using the novel methods of this invention.

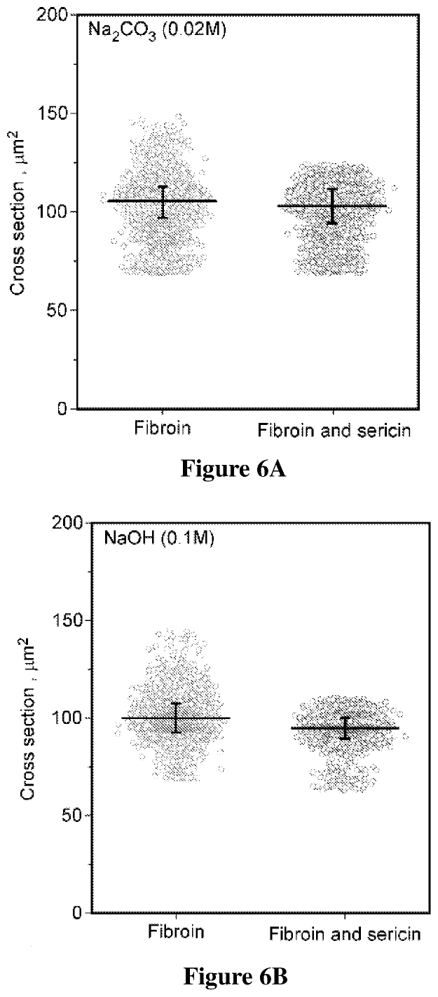

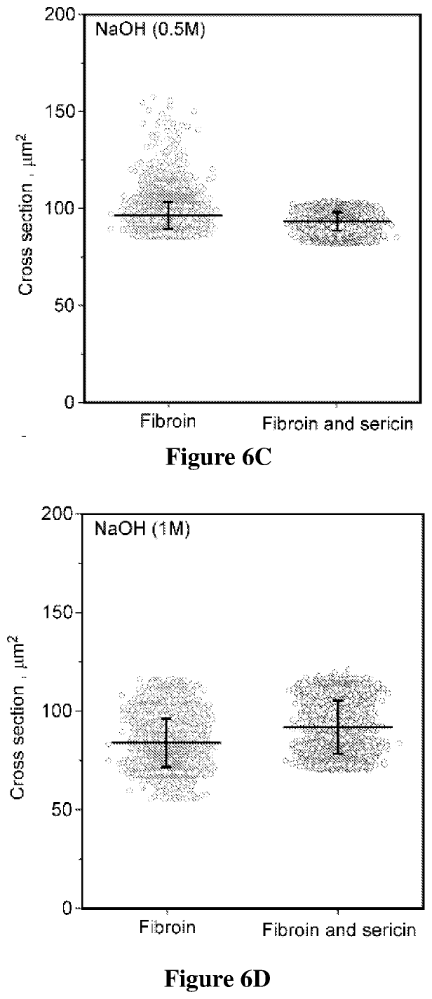

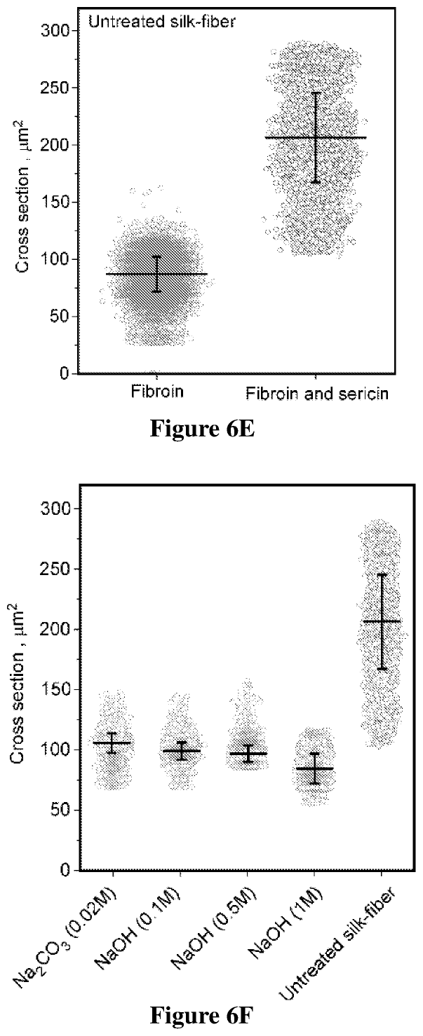

Due to the asymmetric shape and non uniformity of silk fibers, the volumetric parameters are often not used in calculation of silk mechanics. In contrast, in methods of this invention, cross-sectional values are used. To extrapolate cross-sectional parameters for silk fiber components, a post-processing analysis has been applied. The original confocal images are converted into the binary format to calculate the cross-sectional area for each component separately and combined. For this purpose and in some embodiments, a software which integrates over the whole image to obtain cross-sectional areas of fiber components is used. The obtained values for the cross-section of all components, are then used in mechanical calculations as described in detail herein below. The cross-sectional analysis showed inconsistencies and large standard deviation (STD) for fibers that were treated by the standard Na₂CO₃ degumming process (see FIGS. 6A and 6F). In contrast, the narrowest STD was obtained for the novel NaOH 0.5M treatment (FIGS. 6C and 6F). These differences could originate from better solubility of the sericin component in NaOH and/or from the absence of, or the reduction of thermal damage in fibroin protein fibers undergoing this novel treatment process.

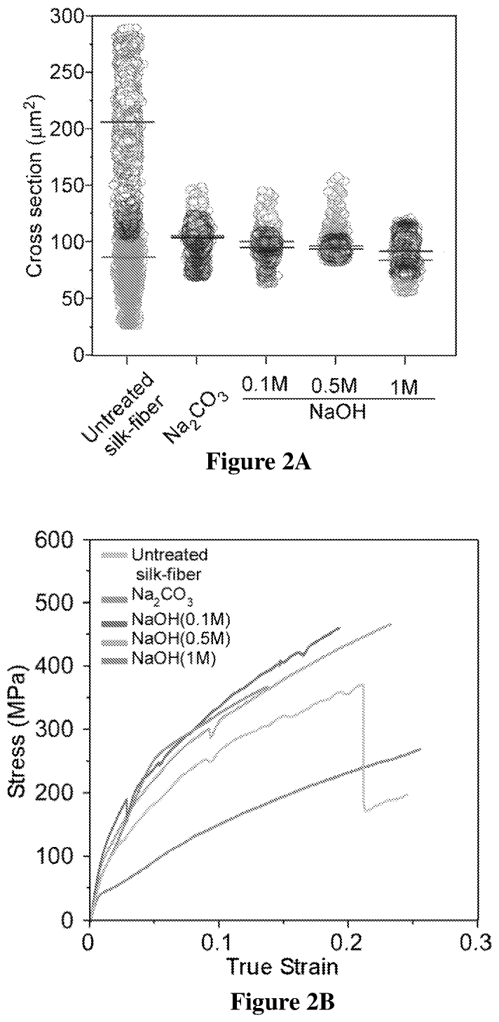

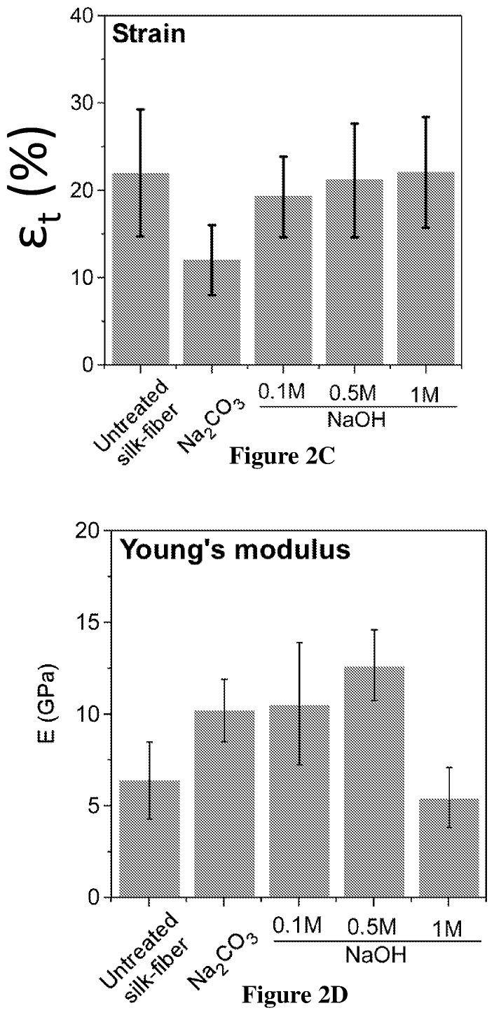

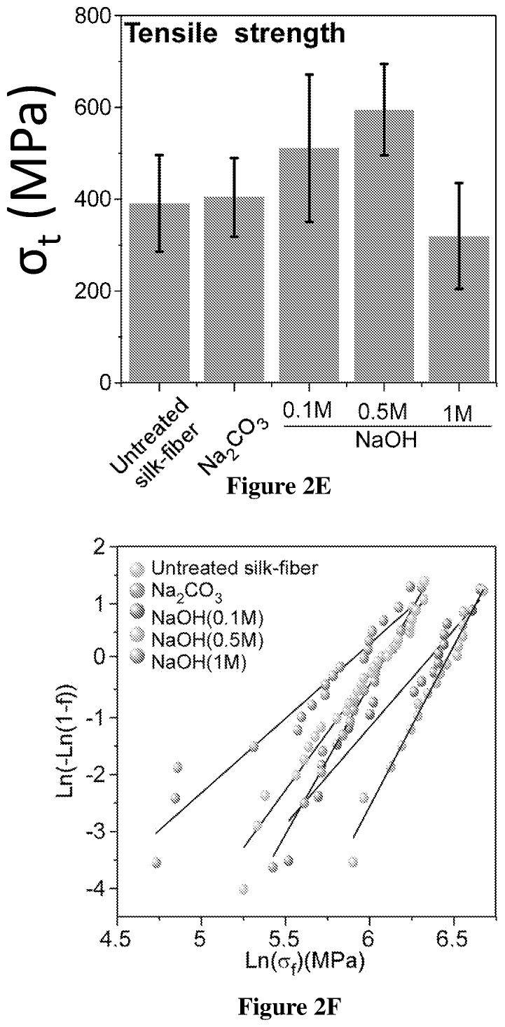

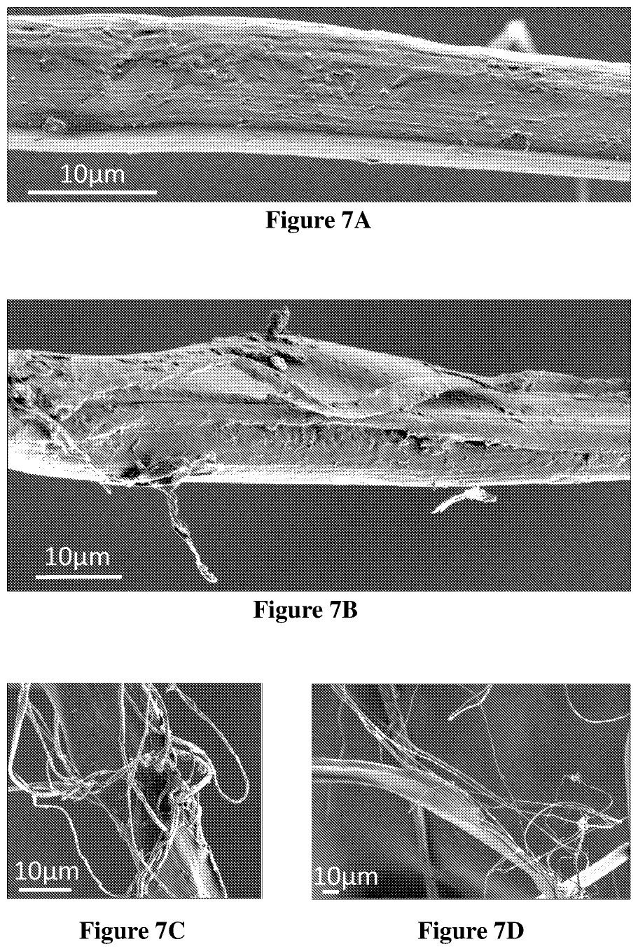

A summary of the cooperative analysis for the measured cross-sectional area ofuntreated fibers, fibers degummed via the standard approach (Na₂CO₃) and silk fibers treated with NaOH-based solution are depicted in FIG. 2A and in FIGS. 6A to 6F. Note that in FIGS. 6A-6D, the cross section is measured for a treated fiber. In these images, the right panel is labelled “fibroin and sericin” and it refers to the fiber after treatment where the fiber includes mainly or totally fibroin, and in some embodiments, small portions of sericin. The left and right panels in FIGS. 6A-6D show similar cross sections because they are taken after treatment and the sericin portion in these treated fibers is either very small or absent. In contrast in FIG. 6E, the right panel shows the fibroin and sericin where no treatment is applied. The left panel in FIG. 6E is also measured for the untreated fiber. It is taken from the cross section of the fibroin only in the untreated fiber as shown in FIG. 1D (top right panel in FIG. 1). Selective Nile-Red dying allows to differentiate the Fibroin from the sericin in this image as detailed herein. The analysis shows that there are substantial differences in the effectiveness of sericin removal when comparing the standard method and the newly-developed NaOH-based process (see FIGS. 6B to 6D). While for the standard protocol, e.g., Na₂CO₃, small traces of the sericin component have been detected, fiber degumming using NaOH did not show any presence of the gum. At high NaOH molar concentrations (e.g., about 1M) microscale bundles decomposed into separated nanofibrils (see FIGS. 7A to 7D, showing various areas along the fiber). Furthermore, analysis of the measured cross-sectional area, which is shown in FIG. 2A and FIG. 6C, revealed that degumming performed in NaOH (0.5M) resulted in the smallest standard deviation (STD) values with almost perfect overlap between the values obtained from confocal microscopy analysis.

Cross-section area measurements of fibroin, and of fibroin and sericin, are conducted before or after treatment as needed. In one embodiment, the (fibroin+sericin) cross section is taken before treatment while the cross section of the fibroin-only is measured for a fiber after treatment. In some embodiments, after treatment, the layer of the sericin is removed and the cross section of fibroin-only is measured. In one embodiment, the fibroin and the (fibroin+sericin) cross section is taken before treatment from an image of a dyed fiber where fibroin and sericin have distinct colors (see details below). In one embodiment, for example, where some sericin is left on the fiber after treatment, a measure of fibroin and left-over sericin (fibroin and sericin) is conducted after treatment.

FIG. 2A represents a calculated cross-sectional area, based on images obtained from confocal analysis and further processing. FIG. 2B represents stress-strain curves of untreated and degummed silk fibers. FIG. 2C is a chart summarizing true strain at break of the untreated and degummed silk fibers from FIG. 2A. FIG. 2D represents a chart summarizing the Young's modulus of the untreated and degummed silk fibers from FIG. 2A. FIG. 2E is a chart summarizing calculated (true) tensile strength of the untreated and degummed silk fibers from FIG. 2A. FIG. 2F is a graphical representation of the calculated two-parameter Weibull distribution plots of the fibers strength.

### Methods of the Invention

In one embodiment, this invention provides a method for removing at least part of a gum layer from silk fibers, said method comprising: incubating said silk fibers in an incubating medium, said incubating medium comprising a hydroxide. In some embodiments the incubating medium consists of one hydroxide and water.

In one embodiment the method is carried out at a temperature ranging between 5 to 60 degrees Celsius (° C.). In one embodiment the hydroxide is selected from: NaOH, LiGH, KOH, RbOH, CsOH, Ca(OH)₂, Sr(OH)₂ or Ba(OH)₂ or a combination thereof. In one embodiment the source of the hydroxide in the incubating medium is: NaOH, LiGH, KOH, RbOH, CsOH, Ca(OH)₂, Sr(OH)₂, Ba(OH)₂ or combination thereof. In one embodiment the concentration of the hydroxide in the incubating medium ranges from between about 0.1M to 1M. In one embodiment the concentration of the hydroxide in the incubating medium ranges from between about 0.05M to 0.8M. In one embodiment the concentration of the hydroxide in the incubating medium ranges from between about 0.4M to 0.8M. In one embodiment a single incubation step is carried out for degumming wherein the incubation medium consists of one hydroxide and water. In one embodiment a single incubation step is carried out for degumming wherein the incubation medium comprises at least one hydroxide and water.

In one embodiment, the incubating medium is a solution comprising a hydroxide. In one embodiment, the solution is an aqueous solution. In one embodiment, the solution comprises water and a base. In one embodiment, the base comprises a hydroxide. In one embodiment, the base is a strong base. In one embodiment, the incubating medium comprises water, hydroxide negative ions and positive metal ions. Hydroxide is referred to as (OH) in some embodiments. In some embodiment, the ionic material (e.g. NaOH) which is the source of the hydroxide is referred to for simplicity as a hydroxide. In one embodiment, the incubating medium further comprises at least one buffer. In one embodiment the incubating medium further comprises at least one salt. In one embodiment the incubating is carried out from between 1 minute to 60 minutes. In one embodiment the incubating is carried out from between 5 minutes to 40 minutes. In one embodiment the incubating is carried out for more than 5 minutes. In one embodiment the method further comprises rinsing the silk fibers in a liquid after the incubation. In one embodiment the rinsing liquid is water. In one embodiment the rinsing liquid comprises water or a solution. In one embodiment said rinsing comprises dialysis. In one embodiment the method further comprises drying the silk fibers after rinsing with a liquid. In one embodiment the method further comprises separating the silk fibers into individual fibers. In one embodiment, the method further comprises selecting a single fiber from a bundle of fibers.

### Method for Determining True Mechanical Properties

To determine the true mechanical properties of silk fibers the follow general steps need to be carried out. It is noted that many silk fibers comprise asymmetric structures and many contain defects (for example fractures at the surface). The present invention facilitates the determination of a cross-sectional area despite the non-uniform nature of the fibers. In principle, this type of calculation can be extended to any fiber, even those that are non-biological, as explained herein. Establishing an accurate measurement for the cross sectional area is crucial for determining mechanical properties and characteristics.

A sample is prepared, to be imaged, typically in confocal microscopy. As such, the silk fiber may be dyed for enhanced detection in microscopy such as confocal microscopy. Cross-sectional images are then collected along the length of the fiber using a confocal microscope. Imaging parameters are typically optimized to ensure high contrast of cross-sections at different focal planes. Imaging parameters such as laser power, wavelength, gain, exposure time and focus are selected to optimize the image quality. Typically, given the structure of silk fibers, an image is optimized such that the various regions in the cross section are easily distinguishable. A stack of images are collated, and software is used for image processing. In different embodiments the z-axis (i.e., axis along the length of the fiber) increments are selected to optimize for image processing. In some embodiments the z increments are small, and the total cross sectional area is integrated along the length of the silk fiber. Ideally, these increments are small. The software measures the cross-sectional area of each image and integrates the cross-sectional area along the whole length of the silk fiber to obtain the volume.

In one embodiment, this invention provides a method for determining average mechanical properties of a plurality of asymmetric fibers, the method comprising:
a) using a confocal microscope to obtain a plurality of cross-sectional images along the length of one of the asymmetric fibers;
b) calculating cross-sectional area of the asymmetric fiber;
c) calculating an average cross-sectional area along the length of the asymmetric fiber;
d) measuring the extension versus load for the asymmetric fiber until the asymmetric fiber breaks;
e) using the average cross-sectional area to obtain a stress-strain plot for the asymmetric fiber;
f) obtaining maximum strength and Young's modulus from the stress-strain plot for the asymmetric fiber;
g) performing stages ‘a’ to ‘f’ for a plurality of asymmetric fibers to obtain a plurality of stress/strain data sets;
h) calculating an average maximum strength and average Young's modulus for the plurality of asymmetric fibers.

In one embodiment the stress-strain plot is calculated using the true stress (at) and true strain (Ft) using the following equations:

$${σ}_{t} = \frac{Pl}{{A}_{0} {l}_{0}} = {σ}_{e} ( 1 + {ε}_{e} ) \qquad eq . 5$$

$${ε}_{t} = {\int }_{{l}_{0}}^{l} \frac{dl}{l} = ln ( \frac{l}{{l}_{o}} ) = ln ( 1 + {ε}_{e} ) \qquad eq . 6$$

In turn, the true Youngs modulus is also determined using the true stress and true strain.

The Weibull parameter is a statistical parameter that is used to characterize the strength, failure, and reliability of a material such as a silk fiber. It provides a statistical distribution of strength of the fibers within a collection of such fibers. In one embodiment, the method further comprises utilizing a two-parameter Weibull distribution, wherein the method comprising;
a) ranking the maximum strength for the plurality of stress/strain data sets from lowest to highest and assigning the data set a number;
b) calculate the natural log of the maximum strength for each the data set;
c) calculate the probability of failure, f(σ), for each of the data sets;
d) plot the natural log of the maximum strength versus ln(−ln(1−f(σ));
e) extract the shape parameter, β, from the gradient and the scale parameter, α, from α=exp(intercept/f) from the plot in ‘d’.

In one embodiment, the shape parameter β and the scale parameter α provides a measure of the mechanical properties of the fiber. In one embodiment, the value of the shape parameter β and of the scale parameter α, gives an indication of the strength of a fiber material, information which is valuable for a user of the material. In one embodiment, the Weibull parameters provide important information for a user of a fiber in view of the various applications of the fiber or components thereof. In one embodiment, the novel cross-section evaluation technique presented herein, enables to calculate the Weibull parameters easily and accurately. In one embodiment, this accurate calculation of the Weibull parameters enabled by methods of this invention, provides essential evaluation of the fibers mechanical properties. Such evaluation is critical in order to decide if the fibers fit a certain application in some embodiments.

In one embodiment, the asymmetric fiber is stained with a dye prior to step (a), the step of: (a) using a confocal microscope to obtain a plurality of cross-sectional images along the length of one of the asymmetric fibers.

In one embodiment, the asymmetric fiber is of biological origin. In one embodiment the asymmetric fiber is degummed. In one embodiment the asymmetric fiber is coated with an additional material. In one embodiment the asymmetric fiber is not degummed.

In one embodiment the fiber is a of biological origin. In one embodiment, the fiber is a natural fiber. In one embodiment, the fiber is synthetic. In one embodiment the fiber is organic. In one embodiment the fiber is inorganic. In one embodiment the fiber is a polymer. In one embodiment the fiber comprises organic and inorganic materials. In one embodiment the fiber comprises silicone. In one embodiment the fiber comprises metal ions. In one embodiment the fiber is sourced from spiders. In one embodiment, the fiber is sourced from any of the following: hymenoptera (bees, wasps, and ants), silverfish, mayflies, thrips, leafhoppers, beetles, lacewings, fleas, flies, midges, arthropods, arachnids and spiders. In one embodiment the fiber is sourced from mammals. In some embodiments the fiber comprises hair or fur.

### Fibers of the Invention

In one embodiment, this invention provides a degummed silk fiber comprising a core and a gum layer wherein the gum layer comprises 0% to 20% of the cross-sectional area of the degummed silk fiber. In one embodiment the degummed silk fiber does not comprise any gum layer. In one embodiment the fiber comprises a negligible amount of gum layer. In one embodiment the gum layer comprises less than 1% of the cross-sectional area of the degummed silk fiber. In one embodiment the gum layer comprises less than 0.01%, or less than 0.1%, or less than 0.5%, or less than 5%, or less than 10%, of the cross-sectional area of the degummed silk fiber.

In one embodiment, this invention provides a degummed silk fiber comprising a core and a gum layer wherein the gum layer comprises 0 wt % to 20 wt % of the degummed silk fiber. In one embodiment the gum layer comprises less than 1 wt % of the degummed silk fiber. In one embodiment, the gum layer comprises less than 0.01 wt %, or less than 0.1 wt %,or less than 0.5 wt %, or less than 5 wt %, or less than 10 wt %, of the degummed silk fiber. Removal of the gum layer can be complete in some embodiments, or incomplete in other embodiments.

In one embodiment the degummed silk fiber exhibits a Young's modulus of at least 10 GPa. In one embodiment the degummed silk fiber exhibits a Young's modulus of at least 10 GPa. In one embodiment the degummed silk fiber exhibits a Young's modulus of between 5-15 GPa. In one embodiment the degummed silk fiber exhibits a Young's modulus which is at least 1.5 greater than the Young's modulus for an untreated silk fiber. In one embodiment the degummed silk fiber exhibits a Young's modulus which is at least 2 times greater than the Young's modulus for an untreated silk fiber. In some embodiments the term “Young's modulus” and “true Young's modulus” are used interchangeably.

In one embodiment the degummed silk fiber exhibits a tensile strength of at least 400 MPa. In one embodiment the degummed silk fiber exhibits a tensile strength of 400 Mpa. In one embodiment the degummed silk fiber exhibits a tensile strength of at least 300 Mpa. In one embodiment the degummed silk fiber exhibits a tensile strength of at least 500 Mpa. In one embodiment the degummed silk fiber exhibits a tensile strength of at least 550 Mpa. In some embodiments the term “tensile strength” and “true tensile strength” are used interchangeably.

In one embodiment, the degummed silk fiber exhibits a strain at break of at least 0.2. In some embodiments the term “strain at break” and “true strain at break” are used interchangeably. In one embodiment the degummed silk fiber exhibits a strain at break of at least 0.3. In one embodiment the degummed silk fiber exhibits a Weibull characteristic strength parameter (α) of at least 400 MPa.

In one embodiment the degummed silk fiber exhibits a Weibull characteristic strength parameter (α) of at least 1.2 times greater than the parameter for an untreated silk fiber. In one embodiment the degummed silk fiber exhibits a Weibull shape parameter (β) of at least 4. In one embodiment the degummed silk fiber is produced by a method as described hereinabove.

In one embodiment, the length of the fiber is in the micrometer range. In one embodiment the length of the fiber is in the centimeter range. In one embodiment the length of the fiber ranges between 1 μm and 10 cm. In one embodiment the length of the fiber ranges between 1 mm and 10 cm. In one embodiment, the length of the fiber ranges between 1 mm and 4 cm. In one embodiment the fiber comprises nanofibers. In one embodiment, the thickness of the fiber is ranging between 1 μm and 50 μm. In one embodiment, for asymmetric fibers, the thickness may have different values in different directions measured along a cross section area of the fiber. In one embodiment, such varied thickness values range between 1 μm and 50 μm. Fibers having thicknesses smaller than 1 μm or larger than 50 μm are also included in embodiments of this invention. In some embodiments, methods of this invention as described herein are applicable to fibers with thicknesses smaller than 1 μm or larger than 50 μm. In some embodiments, methods of this invention as described herein are applicable to fibers with thickness values ranging between 1 μm and 50 μm. In one embodiment, degumming methods of this invention are applicable to coated fibers of any thickness. In one embodiment, methods of evaluating average cross-section of fibers as described herein, are applicable to any fiber having a thickness that can be measured using microscopy. In one embodiment the method further comprises spinning the degummed silk fiber into a yarn or woven into a mat or fabric.

### Reconstituted Silk Fibroin Materials of the Invention

In one embodiment, this invention provides a reconstituted silk fibroin (RSF) material, in the form of solution, the material comprising:
silk fibroin soluble proteins sourced from a degummed silk fiber as described hereinabove; and
a liquid.

In one embodiment the liquid is water. In one embodiment the liquid comprises water. In one embodiment the reconstituted silk fibroin material comprises small protein molecules. In one embodiment the liquid further comprises any of the following selected from: buffer, salt, detergent and reducing agent or any combinations thereof.

### Definitions

As used herein “gum layer” refers to a layer that coats the silk fiber. In one embodiment, this coating layer comprises sericin. In one embodiment the coating layer is asymmetric. In one embodiment the gum layer coats only a portion of the fiber. In one embodiment the gum layer surround at least one fibroin core. In one embodiment the silk fiber comprises two fibroin segments as shown in FIG. 1D. In one embodiment the silk fiber comprises at least one fibroin core coated by sericin. In one embodiment, the gum layer coats each of the two fibroin segments. In one embodiment, the gum layer surrounds each of the two fibroin segments. In one embodiment, the gum layer surrounds the two fibroin segments. In one embodiment, there is only one fibroin segment, and the gum layer surrounds this one fibroin segments. In one embodiment, the gum layer is present between the two fibroin segments. In one embodiment, the gum layer surrounds the fibroin segment(s) and is also present between the two fibroin segments. In one embodiment, the gum layer coats only a portion of a certain cross-sectional area of a fibroin segment. In one embodiment, along the length of a fiber, the extent of the gum layer coating varies.

As used herein “reconstituted” refers to a solution comprising components of degummed fibers. In one embodiment “reconstituted” refers to a solution comprising components of fibers. In some embodiments, reconstituted silk fibroin refers to a liquid which comprises individual silk fibroin proteins and water. In one embodiment the reconstituted silk fibroin is in the form of a suspension. In one embodiment the solution is transparent. In one embodiment the proteins in the solution are completely soluble.

As referred to herein an “incubating medium”, “incubation medium”, “incubation solution”, “incubating solution”, “medium” or “solution” refers to a liquid which comprises at least one substance into which the fibers, or components thereof, are placed.

In one embodiment, as referred to herein “hydroxide” is a diatomic anion with chemical formula OH—, comprising an oxygen atom and a hydrogen atom held together by a single covalent bond.

As used herein the term “asymmetry” refers to any structure that is not perfectly symmetrical. In some embodiments, “asymmetry” refers to any structure that is not uniform in part, or fully. In some embodiments “asymmetry” refers to a structure that comprises defects. In some embodiments the term “asymmetry” can be used interchangeably with the terms “non-uniform”, “ununiform”, “uneven” and/or “irregular” without limiting the scope of the invention.

As used herein the term “load” refers to the applied force in a tensile test, measured in Newtons. As used herein the term “extension” refers to the amount an object increases in length, measured in meters. As used herein the terms “tensile strength”, “maximum strength” and “strength” are interchangeable. Tensile strength is the maximum stress that a material can withstand while being stretched or pulled before breaking. In some embodiments, tensile strength is measured in Pascals, Pa, or Mega Pascals, MPa.

As used herein “stress” (Pa) is a physical quantity that expresses the internal forces that neighboring particles of a continuous material exert on each other, while “strain” (dimensionless) is the measure of the deformation of the material. In some embodiments stress is calculated as the force divided by the cross-sectional area of the material under a load. In some embodiments “strain” represents the displacement between particles in the body relative to a reference length.

As used herein the term “Youngs modulus” (Pa or GPa) refers to a mechanical property that measures the tensile or compressive stiffness of a solid material when the force is applied lengthwise. In some embodiments it is defined as the stress divided by the strain.

As used herein the “shape parameter”, β (dimensionless), is a parameter extracted from Weibull distribution analysis which depends on the defect sizes and is a measure of the variability of the strength (higher β values mean lower strength variability) and hence the fiber reliability.

As used herein the “scale parameter”, α (MPa), is the scale parameter of the Weibull distribution, which represents the average or characteristic strength of the fiber according to the distribution which depends on the stress configuration and test specimen size.

As described herein, σ, refers to the stress. More specifically, σₜ refers to the ‘true stress’ as described herein. In some embodiments, any reference to the stress refers to the true stress. In some embodiments, a represents the strength—how strong the material is under applied stress. In some embodiments, the strength a is taken from the stress-strain plot where stress is represented by a as well. Accordingly, in embodiments of this invention, a represents stress and strength as apparent from the relevant context in certain embodiments, and as known in the art. Similarly, as described herein, F, refers to the strain. More specifically, Ft refers to the ‘true strain’ as described herein. In some embodiments, any reference to the strain refers to the true strain.

### EXAMPLES

### Example 1

### Mechanical Characteristics and Thermal Stability of the Fibroin Fibers

Tensile tests of a single fiber, with multiple repeats, were conducted to measure the impact of different degumming methods on the mechanical characteristics of B. mori silk fibers.

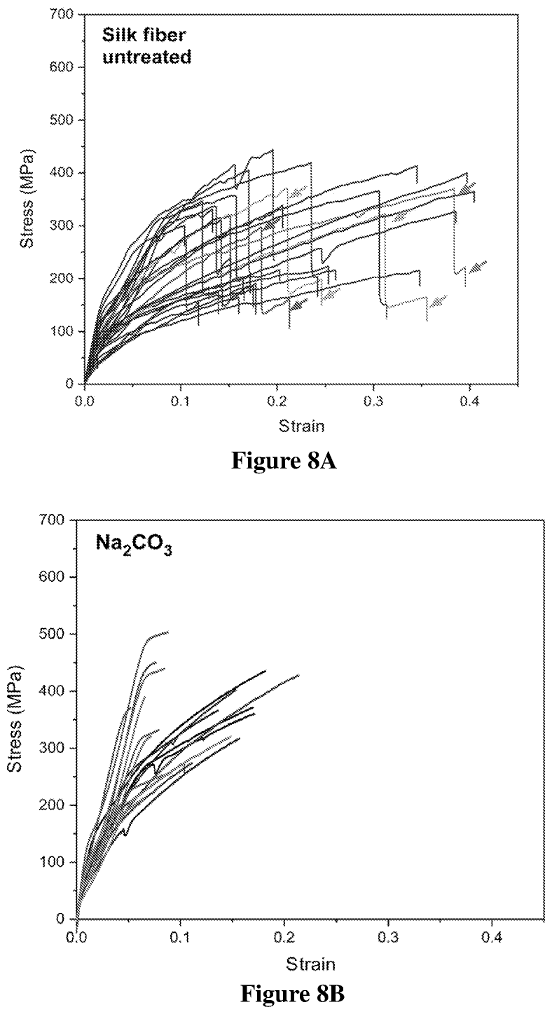

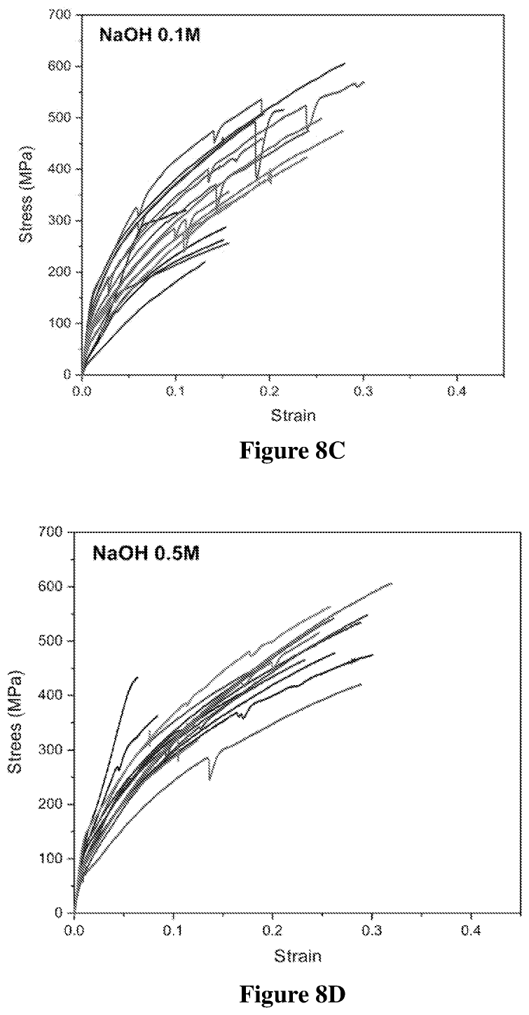

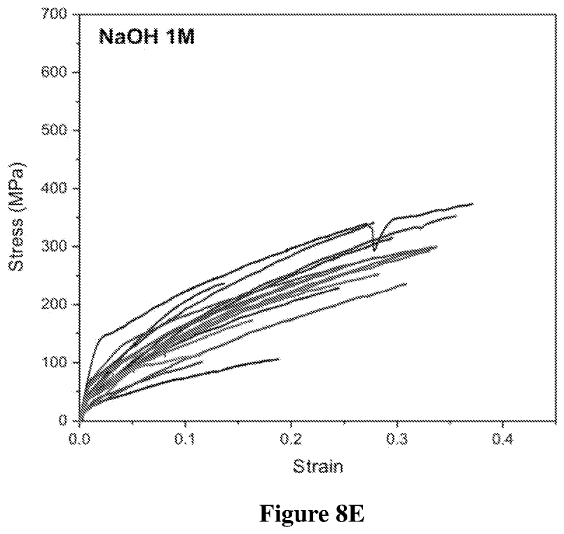

FIGS. 2B to 2F and Table 2 show Young's modulus (E), tensile strength (σₜ), and strain at break (εₜ) of silk fibers that have been subjected to different degumming treatments. The calculations of the mechanical data parameters are based on the measured cross-sectional areas of the fibers before and after degumming treatments, as shown in FIG. 8. Measured and calculated mechanical characteristics, which are summarized in FIG. 2B to 2F and Table 2, show the effectiveness of the different degumming treatments on the structural behavior of silk fibers. Additionally, it serves as a comparison to previous studies where silk fibers were inaccurately considered to have perfectly circular or elliptical cross-sectional shapes.

The tensile stress-strain curves show an initial and concise elastic response (˜1-1.5% strain) followed by a longer plastic behavior for all fibers (FIG. 2B and FIG. 8). The high scattering of the obtained curves (see FIG. 8) is characteristic of imperfect biological materials with low symmetry and presence of defects. After yielding to the applied stress, the fibers exhibited a strain-hardening performance followed by a strain-weakening behavior along with a fluctuation of the stress as they progressively failed.

Table 2 is a tabulated summary of the measured tensile data (mean±SD) for the untreated silk fibers and for the silk fibers degummed using standard, e.g., Na₂CO₃ treatment approaches. Further summarized in this table is the measured tensile data for the herein-developed treatment approaches. In some embodiments the terms “tensile strength”, “maximum strength” and “strength” are interchangeable. Tensile strength is the maximum stress that a material can withstand while being stretched or pulled before breaking.

Several studies on the tensile properties of silk fibers are usually presented in terms of engineering magnitudes, representing only a practical approximation of the true stress and true strain values experienced by the silk fiber rather than a complete analysis of the mechanical capabilities of the material. This is in part because the direct measurement of the instantaneous cross-sectional area is difficult to obtain, or because the validity of the hypothesis of a constant volume of the fibers is not considered in experimental measurements. The mechanical properties of silk fibers are strongly affected by which set of tensile parameters is chosen (true or engineering), as the engineering values could result in a misconception of experiments that combine results from different strain ranges. Introducing the initial actual cross-section area of the fibers (FIG. 2A) into the mechanical calculations is paramount to gain an accurate understanding of the mechanical capabilities of these fibers, as the initial cross-section area has a direct effect on the tensile stress according to the relation σ=F/A₀, where σ is the stress, F is the applied force and A₀ is the initial cross-sectional area of the fiber. Determination of the “true” stress and true strain values of the fibers: The engineering or “nominal” stress and strain values, denoted here as σₑ and εₑ respectively, are obtained from the measured load (P) and displacement (δ) values according to the following relations:

$${σ}_{e} = \frac{P}{{A}_{0}} and {ε}_{e} = \frac{δ}{{l}_{o}}$$

where A₀ is the initial cross-section area of the sample and considered to be constant throughout the test, and l₀ is the initial length of the sample, equivalent to the gauge length of the specimen in the tensile test. When the stress (σₑ) is plotted as a function of strain (εₑ), an engineering stress-strain curve is obtained. This curve should be interpreted with caution, especially beyond the elastic limit, as the sample dimensions experience substantial change from their initial values. To elucidate a more accurate mechanical behavior under tensile stresses of the fibers, one can use the engineering stress and strain to compute the true stress (σₜ) and true strain (εₜ) of the silk fibers. The mathematical calculation of the true stress and true strain is possible under the assumption of volume consistency during stretching. This assumption is valid in the elastic region of the curve because any changes in volume in this region will be very small. Similarly, the assumption is valid in the plastic region because materials, such as silk fibers, are considered to be incompressible during plastic deformation. Furthermore, the true stress (σₜ) and true strain (εₜ) can be mathematically defined as:

$${A}_{0} {l}_{0} = Al \to A = \frac{{A}_{0} {l}_{0}}{l}$$

$${σ}_{t} = \frac{Pl}{{A}_{0} {l}_{0}} = {σ}_{e} ( 1 + {ε}_{e} ) \qquad eq . 5$$

$${ε}_{t} = {\int }_{{l}_{0}}^{l} \frac{dl}{l} = ln ( \frac{l}{{l}_{o}} ) = ln ( 1 + {ε}_{e} ) \qquad eq . 6$$

where A is the cross-sectional area of the sample, dε is the increment of strain, dl is the increment in length and l is the length of the fiber. Considering that the volume of the sample remains constant.

Additionally, considering the events of the significant plastic deformation of silk and the mechanical phenomena observed on such regime, e.g., strain hardening, the determination of the true stress and true strain of silk fibers is of great significance to understanding the actual mechanical capabilities of the material. The justification and calculation of the fibers' true stress and true strain values and their difference to the “nominal” or engineering stress-strain is discussed herein.

Table 2 shows a summary of the measured tensile data for the untreated silk fibers and silk fibers degummed by using standard as well as developed treatment approaches (mean SD).

TABLE 2

| | | | | Weibull parameters on | | |
|---|---|---|---|---|---|---|
| | | | | strength | | |
| Fiber sample | E (GPa) | σₜ (MPa) | εₜ (%) | α (MPa) | β | n |
| Untreated silk-fiber | 6.4 ± 2.1 | 388.1 ± 108.2 | 21.9 ± 7.4 | 426.9 | 4.1 | 28 |
| Na₂CO₃ | 10.2 ± 1.7 | 402.0 ± 88.4 | 12.0 ± 4.0 | 438.6 | 5.3 | 19 |
| NaOH (0.1M) | 10.5 ± 3.4 | 508.1 ± 163.4 | 19.3 ± 4.6 | 584.4 | 3.4 | 17 |
| NaOH (0.5M) | 12.6 ± 2.0 | 591.6 ± 120.6 | 21.2 ± 6.5 | 640.8 | 5.6 | 17 |
| NaOH (1M) | 5.4 ± 1.7 | 316.7 ± 118.9 | 23.1 ± 6.3 | 373.6 | 2.6 | 18 |

The true tensile stress-strain curves, computed using equations (5) and (6), show an initial and concise elastic response (˜1-1.5% strain) followed by a longer plastic behavior for all fibers (FIG. 8). The high scattering of the obtained curves (FIG. 8) is characteristic of the unperfect biological materials with low symmetry and presence of defects. After yielding to the applied stress, the fibers exposed a strain-hardening performance along with a fluctuation of the stress as they progressively failed.

This is attributed to the opening, rearrangement, and gradual breaking of the β-sheets in the fibroin fibers during deformation along the fiber axis. Furthermore, strain-hardening has the purpose of resisting failure and increasing the fiber's mechanical strength, as seen in other biological materials with similar capabilities.

Similar strain hardening/weakening behaviors have been previously observed in spider silk and A. perni silk. Furthermore, strain-hardening has the purpose of resisting failure and increasing the fiber's mechanical strength, as seen in other biological materials with similar capabilities.

In a simple model, untreated B. mori silk fibers are composed of two fibroin fibers enclosed in a sericin shell as discussed herein above and as shown in FIG. 1 (see upper panel “untreated silk fiber”). The mechanical response of each fibroin fiber was observed in the tensile test and pointed with arrows in FIG. 8A. As strain propagated throughout stretching, the stress applied on the fiber increased progressively; when the first fibroin fiber failed, a drop of about half of the original magnitude of the stress was noted. This was followed by a continuing increase of the strain until the catastrophic failure of the second fibroin fiber was completed. The capability of adjacent fibroin fibers, in a single silk fiber, to independently resist different strains can have a positive impact on the toughness of the whole fiber. After the failure of the first fiber, the second fiber can still absorb further quantities of energy through longer plastic deformations. Random drops of stress that were efficiently recovered in the case of all treated fibers, were observed.

The elastic modulus, strength, and strain at break of B. mori obtained from the measurements (FIGS. 2C to 2E) are shown.

Surface treatment of B. mori fibers resulted in increased values of E (FIG. 2D), as after degumming only single fibroin fibers without the existence of the soft sericin shell were tested.

Degumming of B. mori silk with NaOH 0.1M and 0.5M yielded the highest σ values, as shown in FIG. 2E and Table 2, of about ˜420 and ˜480 MPa, respectively. These values are 16%-33% higher than those obtained by the standard degumming treatment with Na₂CO₃ (˜360 MPa). The result suggests that a higher quantity of defects at the fiber surface are produced after degumming silk using the standard protocol, namely boiling in Na₂CO₃-containing solution, affecting the overall strength of the material. Typically, although not absolutely, sericin does not contribute to the strength of the silk. Typically, as shown herein, degumming B. mori silk with NaOH 0.5M resulted in higher strength than degummed silk from A. mylitta, A. pernyi, S. c. ricini, with improvements of between 2%, to 70%.

The strength of fibers is affected by both the defects along the fibers and the non-uniform cross-sectional area. To further understand and correlate the effect of the degumming method on the fiber strength, this invention utilizes a two-parameter Weibull distribution. The theory and justification for using the distribution is discussed below. First, data is ranked, and each data-point (of the strength) is assigned a failure probability and subsequently plotted using equation (3) as shown below, yielding the graph shown in FIG. 2F. Note that “f” and “σ_{f}” in the X/Y labels of FIG. 2F represent the probability of failure of the fiber under applied stress (σ). In equations 1-4 below, f(σ) represents this value. Equation 1:

$$f ( σ ) = 1 - {e}^{( - {( σ / α )}^{β} )} \qquad ( 1 )$$

After equation development as shown herein below, equation 3 is obtained:

$$ln [ - ln ( 1 - f ( σ ) ) ] = βlnσ - βlnα \qquad ( 3 )$$

The left part of this equation is the Y axis in FIG. 2F. The right part of this equation represents a linear graph wherein β it is the slope, ln σ is the ‘X’ and −β ln α is the intercept. See ln(σ_{f}) for the X-axis of FIG. 2F.

The slopes in FIG. 2F indicate the strength distribution widths. The wriggles in the curves of FIG. 2F are commonly found in small sample populations. The data shows that silk degummed with NaOH 0.5M, presented larger β values (intercept not shown in FIG. 2F), a shape parameter, indicating the lower variability of strength compared to Na₂CO₃ and NaOH 0.1M degumming treatments that showed wider distributions of strength by their lower β values (Table 2). The characteristic strength, or scale parameter (α), shown for each sample in Table 2, specifies the distribution location along the x axis of FIG. 2F.

Treatment with NaOH at 1M yielded fibers with lower values of E and a (FIGS. 2D to 2E and Table 2, respectively). This is likely the result of the harsher degumming treatment that may have damaged the surface and the structural integrity of the fibroin fiber (FIG. 2F). Larger deformations were also observed after this aggressive degumming process (similar in some embodiments to those found when boiling the fibers). The crystalline phase, in this case β-sheets, impart dimensional stability to the fibroin fiber, which, in some interpretations, may have been lost due to the degradation of this phase during the harsh degumming process.

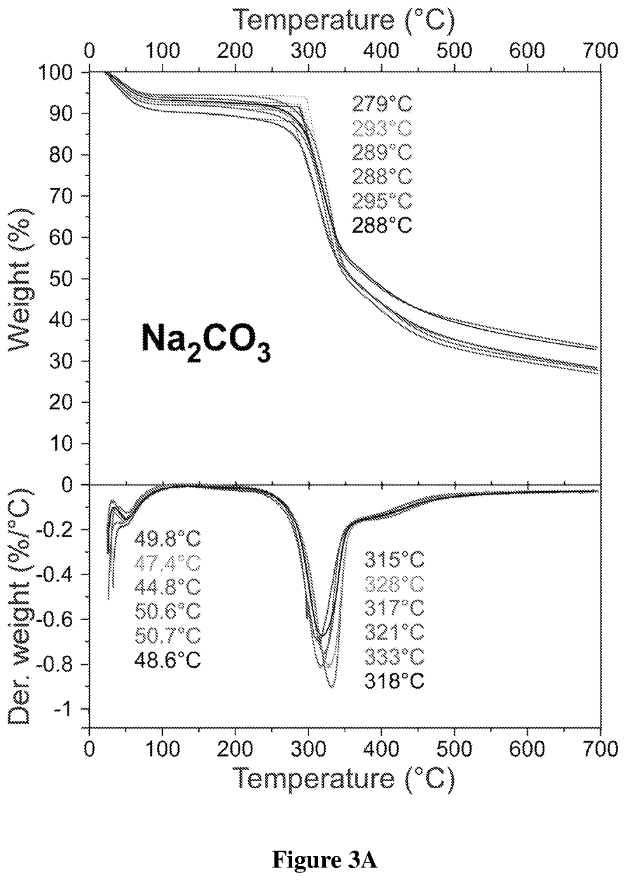

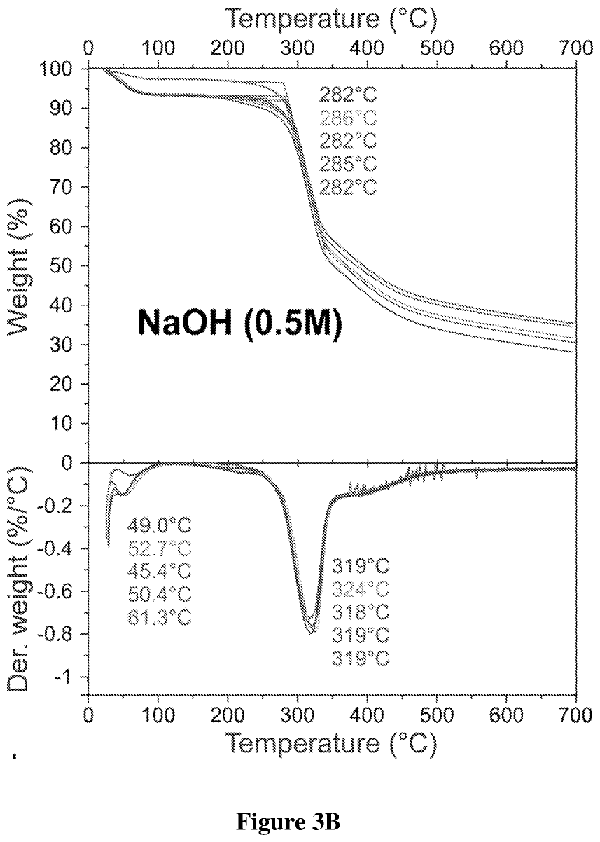

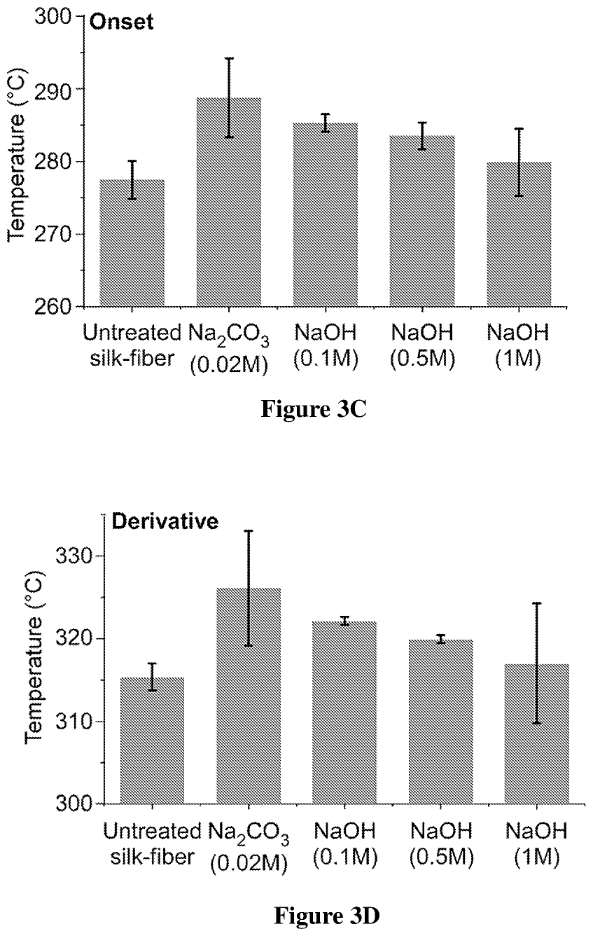

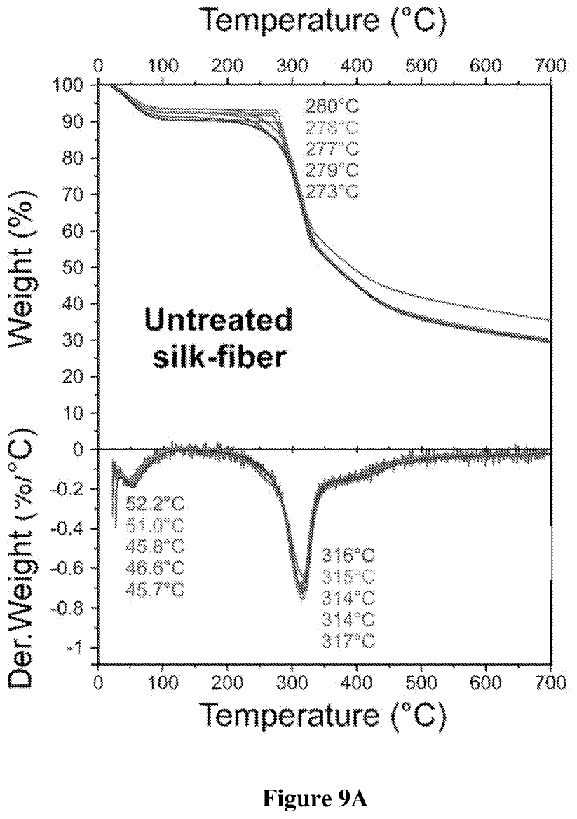

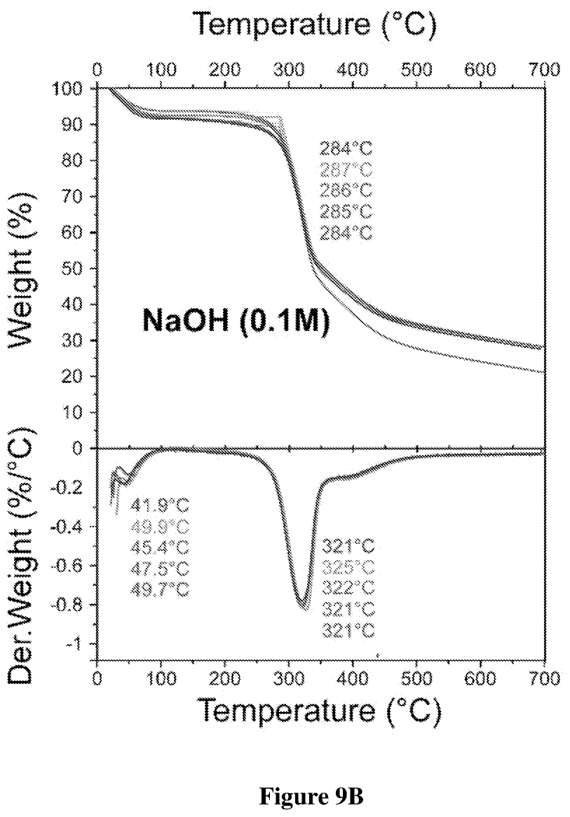

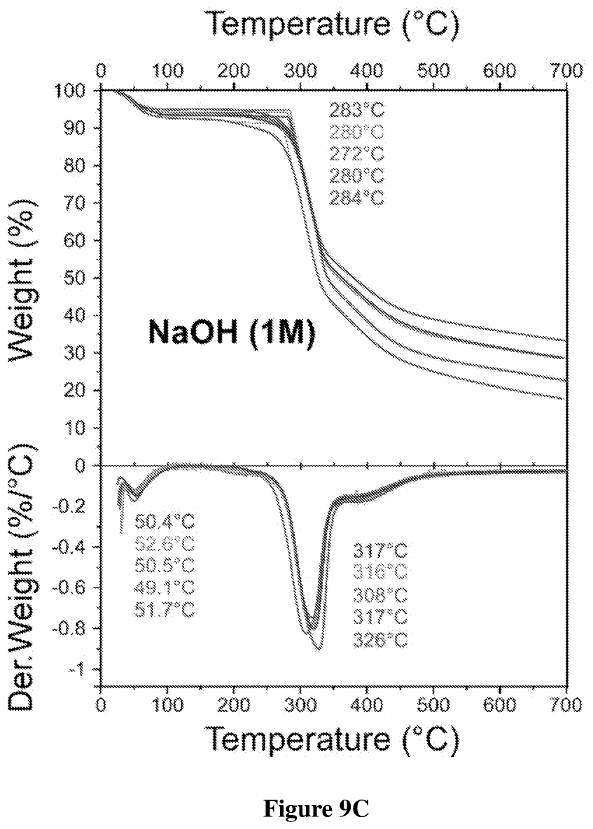

The strain at break of fibroin after degumming the silk with NaOH 0.1M and 0.5M respectively, decreased compared to the strain at break of the silk in its natural form (Table 2). It has been previously shown that stretching the microstructure results in molecular orientation of the β-sheets, yielding a higher degree of crystallinity of the fiber microstructure, and consequently improved mechanical strength and reduced strains. Fiber treatment with 0.1M and 0.5M NaOH is less harsh degumming process compared with the standard protocol (Na₂CO₃). FIG. 2 shows representative examples only, and therefore slightly deviates from the average values in Table 2. The differences in the degumming treatment, in particular with standard Na₂CO₃ protocol as compared with NaOH-based degumming without thermal damage, leads to variations in thermal stability of the fibers. In one example, fibers containing a higher degree of molecular orientation as well as a higher degree of crystallinity tend to show higher thermal stability. To this end, thermogravimetric analysis (TGA) was performed, the results of which are summarized in FIG. 3. FIG. 3A and FIG. 3B illustrate five repeats of TGA scans for degummed silk fibroin fibers in the presence of Na₂CO₃ and with NaOH 0.5M, respectively. FIG. 3C is a graph showing the onset of TGA. FIG. 3D represents the first derivative of TGA curves for degummed fibers with NaOH. The present analysis shows three major peaks of weight loss detected for untreated fibers as well as for fibers subjected to different degumming treatments: 1^{st} peak varying between 40-50° C. (weight loss in 6-8%) associated with a loss of moisture, 2^{nd} peak between 270-300° C. associated with the slow thermal decomposition stage (weight loss 40-45%) and weight loss at the third stage was recorded between 300-350° C. (weight loss 19-22%) which is caused mainly by the breakage of peptide bonds and side groups. Although only small difference has been detected for thermal stability of the degummed silk fibers (see FIGS. 3 and 9 and Table 3) by using the standard protocol and NaOH-based treatment, the major differences were in the reproducibility of the measured values. Thus, for the standard protocol, large variations in weight loss temperature, (at the 2^{nd} peak between 270-300° C.) have been recorded (STD±˜5.53° C.), which likely originates either from a large number of defects or low crystallinity, caused by the standard high temperature thermal treatment. The TGA data recorded from samples degummed in the presence of NaOH were highly reproducible with STD ±˜1.94° C. In some experiments large variations in weight loss temperatures were recorded at STD<˜2.6%. In other experiments the TGA data recorded from samples degummed in the presence of NaOH which was reproducible with STD<˜6%.

TABLE 3
Analysis of weight loss based on the results obtained from TGA measurements.

| | Untreated | Na₂CO₃ | NaOH 0.1M | NaOH 0.5M | NaOH 1M |
|---|---|---|---|---|---|
| water loss I | 7.901 | 6.4315714 | 7.399 | 6.47125 | 6.1372 |
| STD. | 1.160314828 | 1.8005201 | 0.802776121 | 0.157197063 | 0.88616517 |
| weight loss | 39.565 | 43.574143 | 45.781 | 41.6386 | 44.2174 |
| II | | | | | |
| STD. | 1.839938994 | 2.4914833 | 1.889483924 | 2.910990948 | 4.11397105 |
| weight loss | 21.398 | 20.095714 | 20.26 | 19.5525 | 21.586 |
| III | | | | | |
| STD. | 0.999234707 | 1.2060384 | 1.113193604 | 0.728348589 | 1.88493501 |
| weight | 31.136 | 29.898571 | 26.56 | 32.236 | 26.076 |
| remain | | | | | |
| STD. | 2.441440149 | 2.6400658 | 2.948084124 | 3.146510766 | 6.16862059 |

### Example 2

### The Structural Characteristics and the Mechanism of Silk Fiber Degumming

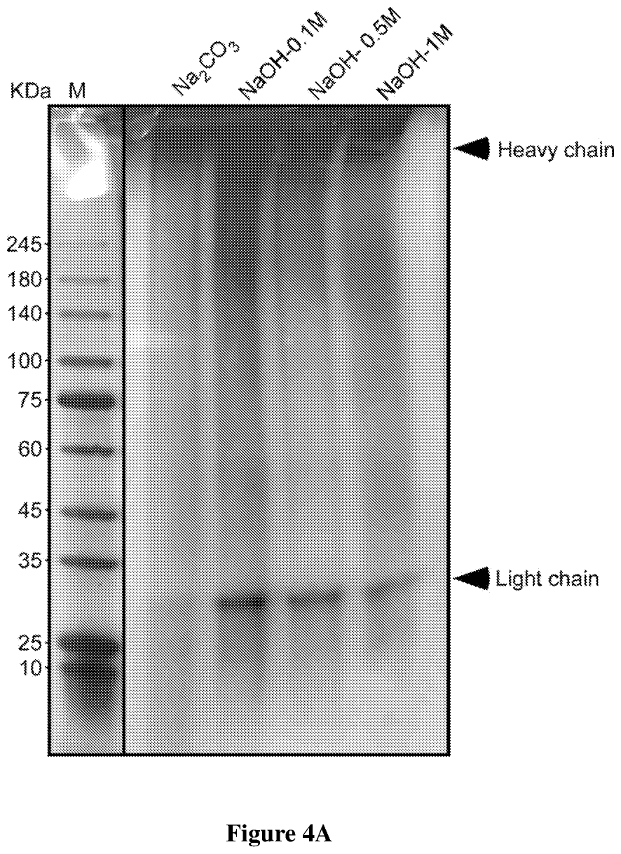

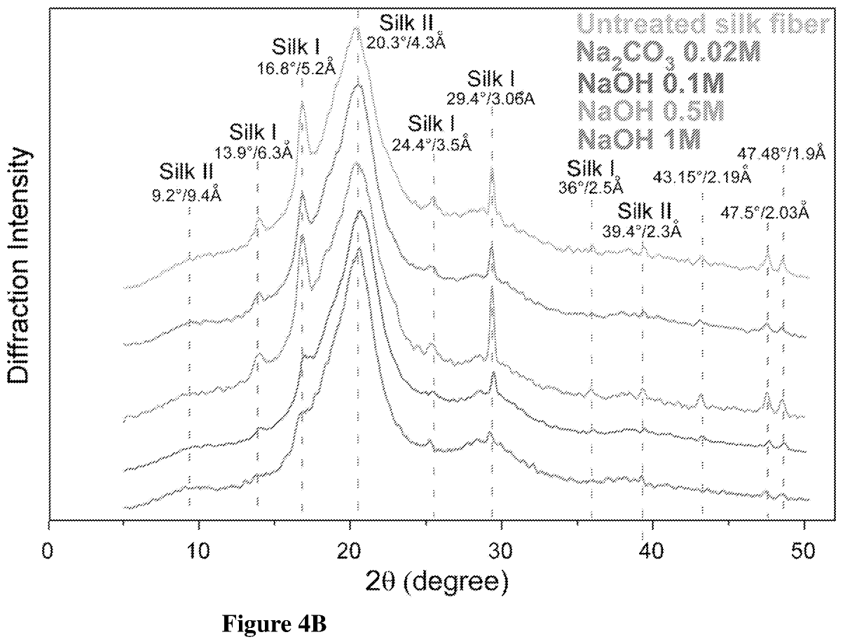

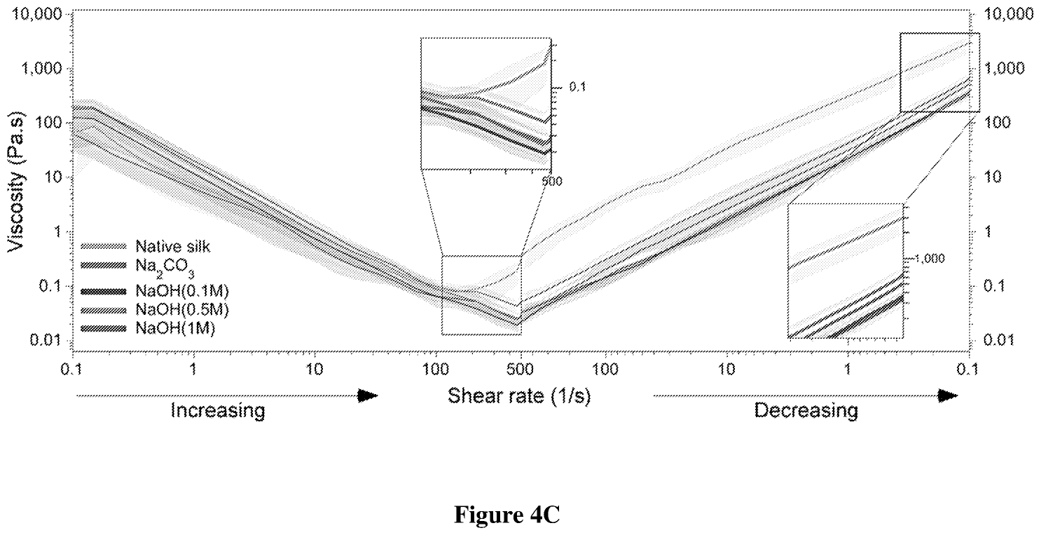

To further understand the structural origin of the increased mechanical strength for NaOH-treated silk fibers, compared to standard treatment, X-ray analysis was performed to probe the differences in the crystalline fraction of the treated fibers. In addition, gel electrophoresis was conducted to examine the integrity of the protein molecules. Generally, silk fibroin protein composing the fibroin fibers occurs in crystalline or amorphous (random coil) form. The fine balance between these two forms (a large fraction of the crystalline component decorated with a small fraction of disordered regions) defines exceptional mechanical characteristics of the fibers. XRD analysis showed the characteristic diffraction peaks of 20 at 9.5°, 20.7° 24.3° and 39.7° (corresponding crystalline spaces are 9.2, 4.3, 3.5 and 2.3 Å, as depicted in FIG. 4B) indicative of the Silk II structure, which is a β-sheet crystalline form, while diffraction peaks of 20 at 12.2°, 19.7° 24.7° (corresponding crystalline spaces are 6.3, 3.67 and 3.5 Å) indicate a Silk I structure, which is a disordered random coil silk protein. The diffraction peaks of 2.5, 3.06, 3.5, 5.2 and 6.3 Å are absent in Na₂CO₃ treated samples, which is indicative of reduction in both crystalline and disordered fraction of the protein. Such event might originate from the loss/disintegration of the protein subunits. Silk fibroin protein is a large globular protein (˜400-450 kDa) composed of two protein subunits, heavy and light chains, which are linked via a single disulfide bridge.

Thus, to further examine the integrity of the fibroin protein molecule as a function of applied treatment, an electrophoretic protein gel analysis has been performed. In detail, a denaturing gel has been used to test the presence of heavy and light chain protein subunits. β-mercaptoethanol, a chemical component utilized in electrophoretic gel analysis, is capable of breaking, via reduction reaction, a disulfide (S—S) bridge, denature the protein and thus, to provide additional information about the protein chain. The results, which are summarized in FIG. 4A, reveal the absence of the light chain subunit in samples degummed using the standard Na₂CO₃-based protocol, while for NaOH-treated samples the presence of both heavy and light chain protein subunits has been observed. This points towards the validity of the NaOH-based degumming method for gentle removal of the gum component without imposing damage on protein units.

FIG. 4A illustrates a gel electrophoresis analysis of silk fibroin protein molecules. FIG. 4B illustrates an XRD analysis conducted on degummed fibers and compared to untreated silk fibroin fibers. Seven main 20 peaks were recorded 10°, 13°, 18°, 20.7°, 25° and 29. FIG. 4C is a rheology analysis of changes in viscosity in response to applied shear, with increasing shear rate from 0.1 s⁻¹ to 500 s⁻¹ and back from 500 s⁻¹ to 0.1 s⁻¹. The following samples were analyzed: native silk fibroin solution obtained from the silkworm and via dissection, RSF solution obtained via chemical re-solubilization of the silk fibers degummed in the presence of Na₂CO₃, NaOH 0.1M, NaOH 0.5M and NaOH 1M. The shade's colors represent the standard deviation.

### Example 3

Rheology of Reconstituted Silk Protein from Degummed Silk Fibroin Fibers and its Structural Hierarchy

To show the effect of different degumming procedures on the rheological behavior of reconstituted silk fibroin (RSF) fluid, changes in viscosity in response to applied shear were measured, with shear rates increasing from 0.1 s⁻¹ to 500 s⁻¹ and decreasing back from 500 s⁻¹ to 0.1 s⁻¹. Furthermore, a comparison was made between rheological characteristics of RSF, obtained via different degumming protocols followed by chemical re-solubilization, and native silk fibroin (NSF) extracted directly from B.mori silk glands via dissection. The viscosity values recorded for RSF solutions obtained following the protocol that includes a degumming step in the presence of 0.5M and 1M NaOH, were significantly higher than those obtained via the standard degumming protocol (FIG. 4C). Such values are comparable to native silk. At the maximum shear rate, (500 s⁻¹), the viscosity for silk solutions reconstituted from fibers degummed with 0.5M and 1M NaOH were 0.03, and 0.05 Pa·s, respectively. However, the viscosity for solutions reconstituted from fibers degummed with 0.1 M NaOH at the shear rate 500 s⁻¹ was 0.25 Pa·s. At the minimum shear rate (0.1 s⁻¹), the viscosity for RSF solutions (degumming steps 0.5M and 1M NaOH) were 501.17, and 644.53 Pa·s, respectively. For the 0.1M NaOH it was 374 Pa·s. In contrast, RSF that derived from degummed fibers with NaOH 0.1M were of higher viscosity than Na₂CO₃ but not significantly. This observation can be explained by insufficient concentration of NaOH for effective removal of the gum layer, that further affects the quality of the RSF solution, similarly to Na₂CO₃.

### Example 4

### Statistical Distribution to Calculate the Strength of Fibers

When applying stress to a stiff material, stress concentrations at local areas emerge because of changes in the specimen geometry, cracks, and surface irregularities, among others. All these manifestations of stress concentrations are observed in silk-worm fibers, where their geometry is constantly changing as formed by the B.mori, and the irregular sericin layer presents micro-cracks along the fiber surface, as shown in FIG. 1. The tensile strength on such fibers differs between different fibers because of dissimilarities in the samples, which can be referred to as defects, and therefore, the strength of silk-worm fibers in the present case cannot be defined by a single value. A statistical distribution is therefore required to quantitively address the dependence of the strength of the fibers on their flaw distribution.

In this regard, the Weibull distribution has been usually considered a suitable statistical model as the distribution considers the lowest possible fracture strength of zero, i.e., the distribution is bounded, it provides accurate failure approximations even with a small population of samples, and the parameters of the distribution allow comparatively superior shape flexibility. To report the strength distribution of a single fiber, the two-parameter Weibull distribution, or Weibull cumulative distribution function, is often used:

$$f ( σ ) = 1 - {e}^{( - {( σ / α )}^{β} )} \qquad ( 1 )$$

where f(σ) is the probability of failure of the fiber under applied stress (σ), α is the scale parameter of the distribution, which represents the average or characteristic strength of the fiber according to the distribution which depends on the stress configuration and test specimen size, and β is the shape parameter which depends on the defects sizes and is a measure of the variability of the strength (higher β values mean lower strength variability) and hence the fiber reliability. To calculate the scale (α) and shape (β) parameters of the distribution a double logarithm of the Weibull equation can be applied as follows:

$$ln ( 1 - f ( σ ) ) = - {( \frac{σ}{α} )}^{β} \qquad ( 2 )$$

$$ln [ - ln ( 1 - f ( σ ) ) ] = βlnσ - βlnα \qquad ( 3 )$$

Then, eq. (3) is plotted yielding a linear graph of slope β. The Weibull scale parameter (α) of the fiber is readily obtained from the intercept of this line, given by:

$$α = {e}^{( intercept / β )} \qquad ( 4 )$$

An example of a two-parameter Weibull distribution of different fibers is shown in the graphical representation of FIG. 2F.

In some embodiments, 6 represents the strength—how strong the material is under applied stress.

Embodiments of the present invention demonstrate the formulation for non-thermal removal of sericin gum layer from silk fibroin fibers. The developed treatment improves fiber mechanics and thermal stability. The method further enables obtaining soluble silk of high purity and rheological characteristics similar to those of native silk. Furthermore, the method disclosed herein provides for the determination of accurate and precise mechanical characteristics of biological fibers such as silk which accounts for the fibers' asymmetry and/or the presence of defects. The developed approaches are beneficial not only for the generation of silk-based materials with tailored properties, but also for correctly determining the mechanical characteristics of asymmetrical fibrous materials made of natural and/or synthetic building blocks. Methods of this invention can be extended to other non-biological fibers.

### Example 5

### Calculations and Measurements for the Weibull Distribution

One way to calculate accurate and precise mechanical properties is by using experimental and analytical methods together. The following is a step-by-step example of how to calculate mechanical parameters of fibers:
1) Use an experimental setup to measure the extension (mm) versus load (N) for a fiber until the fiber breaks. Perform this measurement on a plurality of fibers.
2) For the purposes of comparing degumming processes, stage 1 is carried out for different degummed treatments e.g., at different molarity and incubation medium such as NaOH or Na₂CO₃ at between 0M to 1M.
3) Use a confocal microscope to take cross-sectional images of the fibers along the length of the fiber. Typically, the fiber will be dyed beforehand to distinguish between regions of different materials e.g., fluorescence for materials with different extinction coefficients. As discussed above, the confocal microscope, with subsequent processing, can distinguish between fibroin and sericin regions. For fully degummed fibers, the sericin region will be absent, negligible, or relatively small.
4) A processing unit calculates the cross-sectional area of the fiber as a whole, the fibroin region and the sericin region. Statistical analysis yields the average cross-sectional area of each component of the fiber as well as a standard deviation of the data set.
5) The average cross-sectional area (for any region in the fiber, or the total area) is then used to calculate the tensile stress (σ(MPa)=F/A=Force(N)/Area(m²)) versus strain of each fiber.
6) The maximum strength (MPa) is measured for each fiber using the stress-strain curve. It is the maximum stress that a material can withstand while being stretched or pulled before breaking. The standard deviation of this value is also obtained.
7) The Young's modulus (MPa) is determined from the gradient of the stress-strain curve for each fiber. The Young's modulus is then averaged for all fibers in that data set. The standard deviation of this value is also obtained.

The above defined method can be further improved by using a two-parameter Weibull distribution, a statistical analysis tool, to define the fiber mechanics more accurately and precisely. There are many versions of Weibull distribution which define a statistical analysis tool. Often, using more data points will produce a lower standard deviation and a more precise result of a characteristic, in this case the mechanical properties of fibers.

The following example will demonstrate how the Weibull distribution is used:
collect stress/strain data sets for a plurality of fibers. For the purposes of example, at least five separate stress/strain curves can be used;
From these data sets, as above, extract the maximum strength for each fiber;
Rank the maximum strengths using an index, n, (n=1, 2, 3, . . . ), from lowest to highest with n=1 being the lowest;
Calculate the natural log of the maximum strength i.e., ln(strengthₘₐₓ);
Calculate the probability of failure, f(σ), as follows: f(σ)=(n−0.5)/nₘₐₓ, wherein n is the (ranked) fiber number in the data set and nm is the number with the highest maximum strength;
Plot the natural log of the maximum strength versus ln(−ln(1−f(σ)) which provides a Weibull distribution;
The above plot gives a linear distribution where β is the gradient and α is calculated by α=exp(intercept/β)

### Example 6

### Fibroin Extraction

The extraction of fibroin from Bombyx mori silkworm cocoons was carried out as follows. Silkworm cocoons were chopped and then boiled in 20 mM sodium carbonate (≥99.5%, Fischer Chemical, USA) solution at a ratio of 200 mL solution per gram of raw cocoon. This process yielded degummed fibers. The degummed fibers were then washed and dried, followed by desolvation at 60° C. in a concentrated solution of aqueous lithium bromide. The resultant solution was centrifuged and dialyzed against Milli-Q water. In some embodiments, dialysis was performed in order to remove LiBr from the solution. Following dialysis, the solution does not comprise LiBr in one embodiment.

In some embodiment, following dialysis, the dissolved fiber components were dialyzed again against a salt solution. Such additional dialysis resulted in insertion of desired salt(s) into the reconstituted fibroin solution. In one embodiment, adding salts such as CaCl₂), KCl, NaCl to the reconstituted solution enhances the mechanical properties of the reconstituted solution. In one embodiment, adding salts to the reconstituted solution enhances the mechanical properties of any fiber that is later spun from such solution or any material that is formed by the reconstituted solution. In one embodiment, adding salt to the reconstituted solution increase the viscosity of the solution. This may be advantageous for properties of fibers/materials that are formed from the reconstituted silk protein solution.

In some embodiments, this invention provides a process for preparing a reconstituted fibroin solution, the process comprises at least a portion of the steps described herein above. For example, in one embodiment, salts are added to the reconstituted solution following removal of LiBr. In another embodiment, no salt is added to the reconstituted solution following removal of LiBr. In one embodiment, salts are added using dialysis. In one embodiment, salt(s) are added without using dialysis. In one embodiment, salts are added from solid salt(s). In one embodiment, salt(s) are added from a salt(s) solution. In some embodiments the dialysis is carried out between 1 to 5 hrs. In some embodiments the dialysis is carried out between 5 to 10 hrs. In some embodiments the dialysis is carried out between 1 to 2 days. In some embodiments the dialysis is carried out between 1 to 5 days. In some embodiments the dialysis is carried out between 3 to 4 days.

In some embodiments, reconstituted silk fibroin refers to a liquid which comprises individual silk fibroin proteins and water. In some embodiments the reconstituted silk fibroin is a solution in which the fibroin is completely soluble. In some embodiments the reconstituted silk fibroin is in the form of a suspension. In some embodiments, aggregates of silk fibroin proteins are present in the reconstituted silk fibroin solution. In some embodiments stabilizing agents are added to the reconstituted silk fibroin solution.

In some embodiments, the resulting silk fibroin material is in liquid form. In some embodiments the silk fibroin material is dissolved in water or in a solution such that individual silk fibroin proteins are present in the water or in the solution.

In one embodiment, this invention provides a process of producing silk-protein solution, said process comprising providing silk fibers degummed in a hydroxide solution as described herein, and dissolving the silk fibers in a salt solution.

In one embodiment, the salt solution comprises a salt selected from: LiBr, ZnCl₂, CaCl₂), Ca(NO₃)₂ and LiSCN. In one embodiment, the solvent of the salt solution is selected from water, methanol, ethanol, or any mixture or combination thereof.

In one embodiment, following dissolution of the fibers in the salt solution, the resultant solution is centrifuged and dialyzed against water to remove the salt. In one embodiment, salt removal is complete. In one embodiment, only a portion of the salt is removed.

In one embodiment, following salt removal by dialysis, salts are introduced into the solution again. According to this aspect and in one embodiment, the salt introduced are selected from (but not limited to): CaCl₂, KCl, NaCl, or any combination thereof. In one embodiment, salt addition at this stage enhances mechanical properties of the solution. In one embodiment, the silk-protein solution is a reconstituted silk fibroin.

In one embodiment, prior to dissolution of the degummed fibers in a salt solution, the fibers were used for optical and/or for mechanical properties measurements as described herein. As described herein—hydroxide degumming was conducted instead of sodium carbonate degumming in embodiments of this invention.

### Example 7

### Confocal Microscopy

The 3D images were taken by Zeiss LSM 800 Confocal Imaging System (Carl Zeiss AG, Germany) with a Plan-Apochromat 20×/0.8 M27 (FWD=0.55 mm) objective for confocal imaging. At least five 3D images were taken from each of the samples; native silk fibers and the different treatments of degummed fibers Na₂CO₃, NaOH 0.1M, NaOH 0.5M, and NaOH 1M. Briefly, the samples were incubated with Nile red (with a final concentration of 3 μM) for 16 hours at room temperature, and then placed on glass slides and then covered and sealed with cover slides. The conditions for the images were: Excitation with Led lasers of 559 nm (for Nile red excitation) and 346 nm (for intrinsic fluorescence) and the emission of 636 nm (for the Nile red) and 442 nm (for the intrinsic fluorescence). ˜90 slices (˜33 μm) of Z-stacks were taken and the resolution was around 0.105×0.105×0.37 μm/pixel. The 3D images were reconstructed by using Imaris software. In addition, the 3D images were post-processed by script of Matlab software to calculate the cross-section of the fibers. FIG. 12 shows a schematic representation of z-stacking along the fiber.

### Example 8

### Tensile Test

The following provides an example of an experimental setup for a tensile test. Generally, tensile tests are carried out by securing a sample at two ends under a controlled tension until failure. In this case a fiber is secured at either end, e.g., with clamps, wherein the clamps are moved apart at a controlled speed until the fiber tears, fractures, breaks and/or ruptures. The experimental setup can vary from experiment to experiment. In some embodiments the experimental setup can be a system comprising mechanical and electrical apparatus, computers, processing units and display units that measure the extension of a thread versus an applied load. In further embodiments, such a system can input and output data related to the mechanical properties of the measured fiber and/or material. In some embodiments the experimental setup measures the mechanical properties of more than one fiber.

Depending on the nature of the measured sample, some of the following mechanical properties can be measured and obtained: Young's modulus, Poisson's ratio, yield strength, strain-hardening, rupture, necking, ultimate strength, etc.

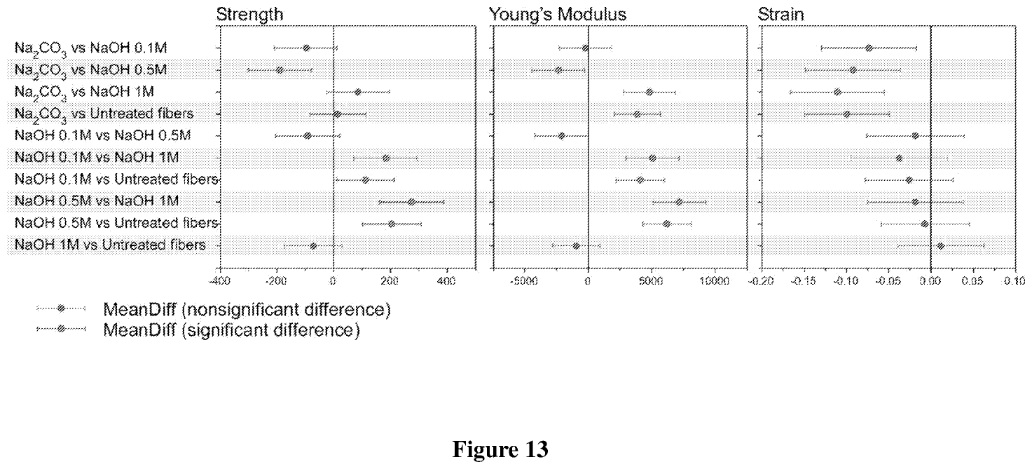

Single fiber specimens for the tensile test were prepared by gluing the untreated and treated silkworm fibers, produced by Bombyx mori, on windowed paper frames with a gauge length of 20 mm. The samples were taken from the middle layers of three different Bombyx mori cocoons. Quasi-static tensile tests of the single fibers were conducted with an Instron 5965 universal testing machine (UK) equipped with a 10N load cell, at a strain rate of 1 mm/min at room temperature. Fiber clamps were used to hold the paper frame on the instrument, and prior to testing, the side edges of the frame were cut out. A minimum of 17 fibers were tested for each sample as specified in Table 2, and tensile properties (Young's modulus, strength, and strain at break, etc.) were calculated from the raised stress-strain curves according to the cross-sectional area of each fiber as obtained in FIG. 6. The plots were computed in the form of true stress-true strain curves according to the cross-sectional area of each fiber set, as obtained in FIG. 2A, and tensile properties (Young's modulus (E), tensile strength (σₜ), and strain at break (εₜ)) were calculated. All quantitative mechanical results were expressed in terms of mean±standard deviation (SD). One-way analysis of variance (ANOVA) was conducted using OriginPro 2022 software to determine whether significant differences existed among the mean values of the experimental groups as shown in FIG. 13. Differences between groups was statistically significant at p<0.05.

### Example 9

### Bulk FT-IR Spectral Measurements

Fourier-transform infrared (FT-IR) spectra for the bulk regenerated fibroin were obtained using a Nicolet iS50 FT-IR spectrometer equipped with an ATR Smart iTX (attenuated total reflectance) accessory with a resolution of 4 cm⁻¹ and 32 individual scans for each measurement.

At least three measurements were taken for each sample, and the spectra were normalized and averaged. Seven peaks were selected for the fitting analysis; intermolecular β-sheet (1609, 1621 and 1631 cm⁻¹), α-helix/random coil (1650 cm⁻¹), β-turn (1673 cm⁻¹) and for antiparallel amyloid β-sheet (1695 and 1703 cm⁻¹).

### Example 10

### Analysis of IR Spectra

All the IR spectra (bulkFT-IR and nano IR) between ˜1720-1600 cm⁻¹ were linear baselined to cover the amide I region. To resolve the secondary structures of the samples, the spectra were normalized and averaged. Then the spectra were fitted (by OriginPro 2019b 64 bit software) by selecting seven Gaussian peaks (1609, 1621, 1631, 1650, 1673, 1695 and 1703 cm⁻¹ with a freedom of 2 cm⁻¹). The fitting analysis for all the spectra have reached to fit converged, and Chi-Sqr tolerance value of 1E⁻⁶. The secondary structures interpretation of these peaks 1609, 1621 and 1631 cm⁻¹ for intermolecular β-sheet, 1650 cm⁻¹ for α-helix and random coil, 1673 cm⁻¹ β-turn and 1695 and 1703 cm⁻¹ antiparallel amyloid β-sheet.

### Example 11

### High-Resolution Scanning Electron Microscopy (HRSEM) Analysis

HRSEM images were obtained using Ultra-55 and SIGMA Ultra-high-resolution SEM (Carl Zeiss, Germany). The samples were placed onto aluminum stubs and fixed with a carbon tape.

### Example 12

### X-Ray Diffraction (XRD) Spectroscopy

XRD of crystalline structures associated with silk I and silk II polymorphisms present in silk fibroin fibers was carried out in reflection geometry using a TTRAX III (Rigaku, Japan) theta-theta diffractometer with a rotating Cu anode operating at 50 kV and 200 mA. A bent graphite monochromator and PMT detector were aligned in the diffracted beam and θ/2θ scans were performed under specular conditions in the Bragg-Brentano mode with variable slits. The 20 scanning range was 1-80 degrees with a step size of 0.025 degrees and a scan speed of 0.4 degrees per minute.

### Example 13

### Gel Electrophoresis (SDS-PAGE)

25 μg of the silk fibroin sample was loaded and run on a gradient gel (4%-20%) from Geba using the manufacture protocol. The gel was stained by InstantBlue® Coomassie Protein Stain (ISB1L) (ab 119211) overnight and washed for several hours with water.

### Example 14

### Silkworm Dissection

Bombyx mori larvae at their fifth instar were anesthetized with N₂ for 15 min and then rapidly dissected by removing the head and applying a longitudinal dorsal incision. Silk glands were gently extracted and rinsed with Mili-Q water and then gently placed on a glass slide (25×16 mm), which was then set on a microscope stage for microscopy detection.

### Example 15

### Rheological Analysis

Rheological characterization was done using HR-20 Discovery Hybrid Rheometer (TA Instruments, US) using an aluminum 40 mm diameter parallel plate geometry at 25° C. The geometry was lowered to a gap of 100 μm at the slowest speed possible. A small amount of distilled water was applied around the specimen and the area was enclosed using a loose-fitting cover, to avoid drying and skin formation. The sample was initially sheared at a constant shear rate of 1 s⁻¹ for 100 seconds to evenly distribute the liquid and get rid of any residual stresses due to previous handling of the sample. Next, an oscillation frequency test was done with strain of 0.02 (within the material's linear elastic region) and angular frequency of 100 rad/s to 0.1 rad/s. Then, the samples were subjected to two repetitive steps of flow sweep tests, each consisting of an increase from 0.1 to 500 s⁻¹ and decrease from 500 to 0.1 s⁻¹. The last step included a second oscillation frequency test with same parameters as before.

### Example 16

### The Structural Characteristics and the Mechanism of Silk Fibers' Degumming

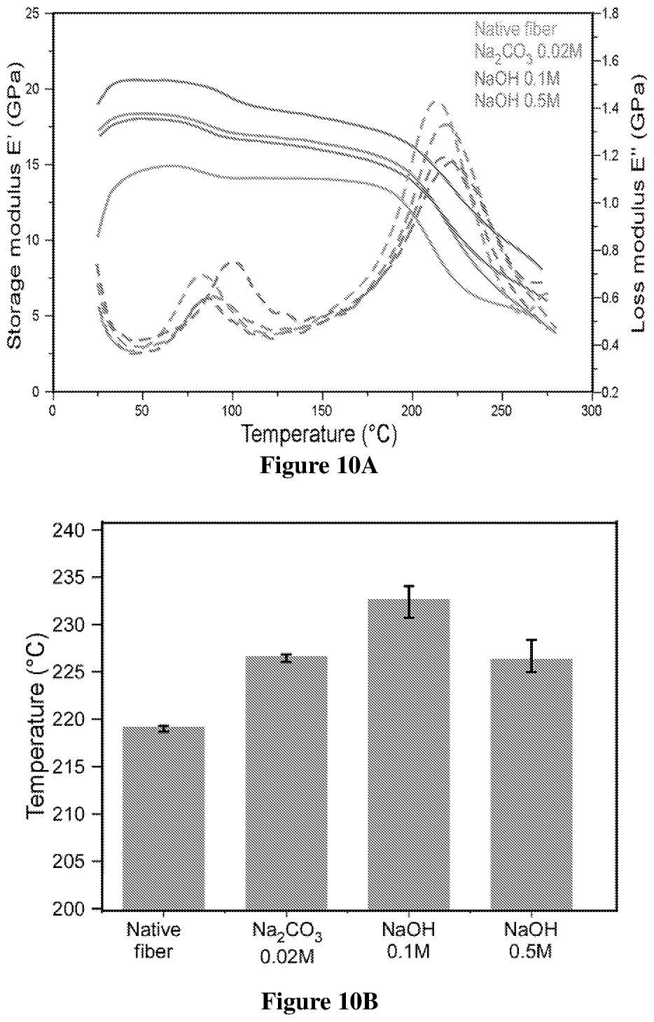

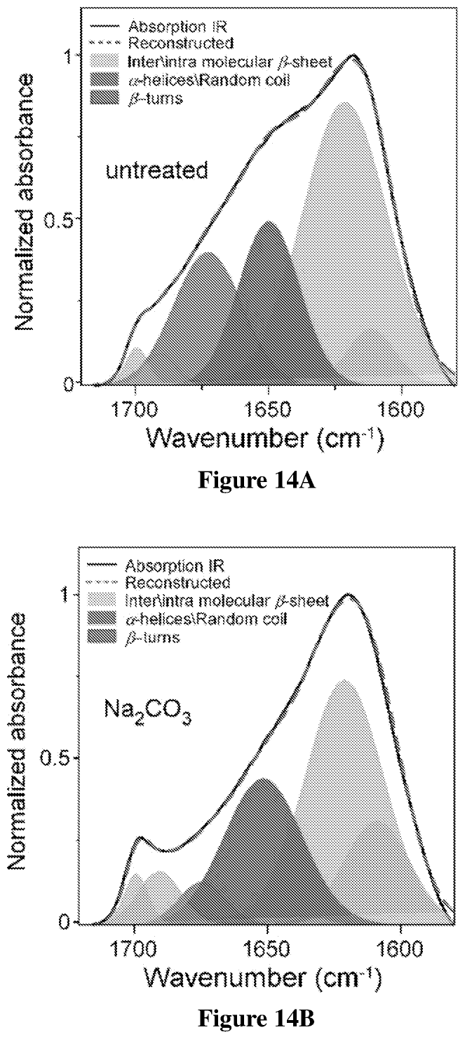

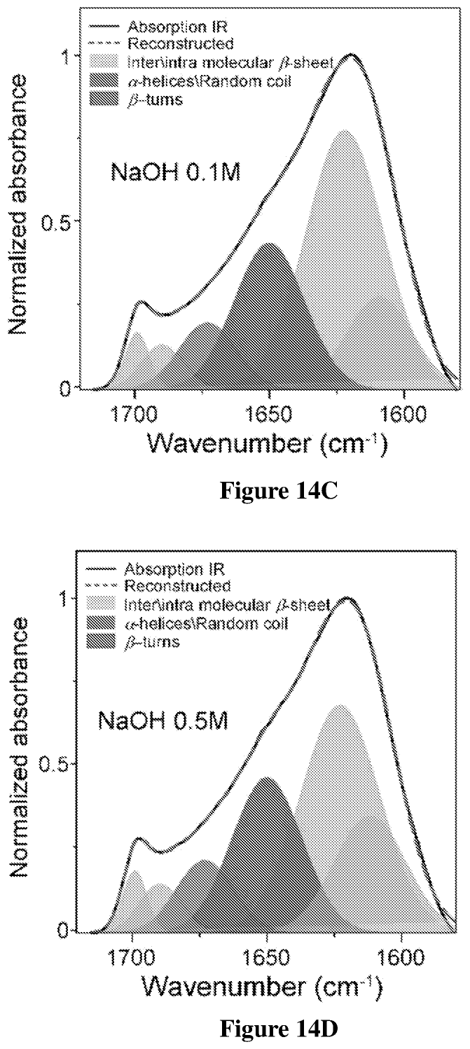

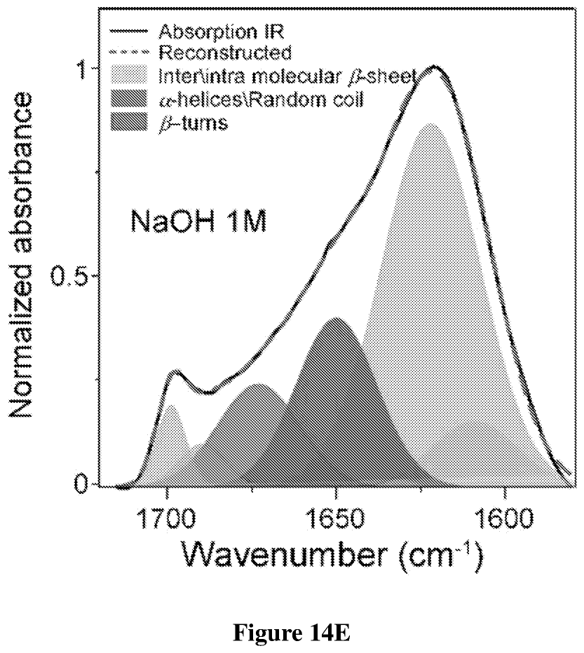

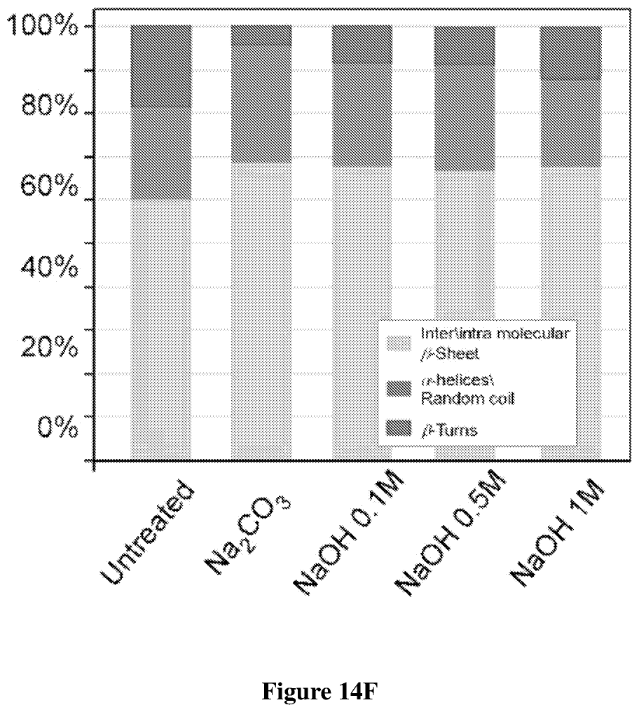

To further understand the structural origin of the increased mechanical strength for NaOH-treated silk fibers, compared to standard treatment, a Fourier transform infra-red spectroscopy (FT-)IR analysis was performed in which the changes in silk protein secondary structure were evaluated. In general, the vibrational spectra of proteins/peptides are characterized by two major bands, namely, amide I (1600-1700 cm⁻¹) and amide II (1480-1600 cm⁻¹), which correspond to C═O and NH bend/CH stretching, respectively. The results summarized in FIG. 14 highlighted the existing variations in between the protein FTIR spectra obtained from degummed fibers. The Na₂CO₃ degummed fibers contained 27% of α-helix, and random coil as well as 4.5% of 3-turn, while the degummed fibers with NaOH procedures contained 20-24% and 8.7-12.4%, respectively. The level of β-sheet in all the procedures of degummed fibers was similar, 66.7-68.7% (FIG. 14). The slight changes in the secondary structure between the degummed fibers led us to test additional features in the level of interaction binding between the fibrils bundle that compose the fiber. Therefore, dynamic mechanical analysis (DMA) was performed to measure the fibers' glass transition (Tg) and crystalline level. The crystalline levels of 0.1M and 0.5M (NaOH) were higher than the standard degumming. Moreover, the T_{g} of 0.1M and 0.5M (NaOH) are at a higher temperature than the standard degumming, which points to the strong hydrogen bonds between amide groups in adjacent protein chains at their amorphous structure. These results suggested that 0.1M and 0.5M degumming fibers contain hydrogen bonds at their amorphous structure, and the β-sheet structures are more stable and stronger (see FIG. 10A, 10B).

X-ray analysis was performed to probe the differences in the crystalline fraction of the treated fibers and gel electrophoresis to examine the integrity of the protein molecules. Generally, silk fibroin protein comprising fibroin fibers mainly occur in crystalline or amorphous (random coil) form. The fine balance between these two forms—a large fraction of the crystalline component decorated with a small fraction of disordered regions—defines exceptional mechanical characteristics of the fibers. XRD analysis showed the characteristic diffraction peaks of 20 at 9.5°, 20.7° 24.3° and 39.7° (corresponding crystalline spaces are 9.2, 4.3, 3.5 and 2.3 Å, as depicted in FIG. 4B) indicative of the Silk II structure, which is a β-sheet crystalline form, while diffraction peaks of 20 at 12.2°, 19.7° 24.7° (corresponding crystalline spaces are 6.3, 3.67 and 3.5 Å 54,56,57) indicate a Silk I structure, which is a disordered random coil silk protein. Interestingly, the diffraction peaks of 2.5, 3.06, 3.5, 5.2 and 6.3 Å were absent in Na₂CO₃-treated samples, which is indicative of a reduction in both crystalline and disordered fractions of the protein.

To further examine the integrity of the fibroin protein molecule as a function of applied treatment, an electrophoretic protein gel analysis was performed. In detail, denaturing gel has been used to test the presence of heavy and light chain protein subunits. β-mercaptoethanol—a chemical component utilized in electrophoretic gel analysis, is capable of breaking, via reduction reaction, disulfide (S—S) bridges, denature protein and thus, providing additional information about the protein chain. The results, summarized in FIG. 4A, revealed an absence of light chain subunits in samples degummed via using standard Na₂CO₃-based protocol, while for NaOH-treated samples the presence of both heavy and light chain protein subunits has been observed. Such an observation points to the validity of the NaOH-based degumming method for gently removing the gum component without damaging protein units.

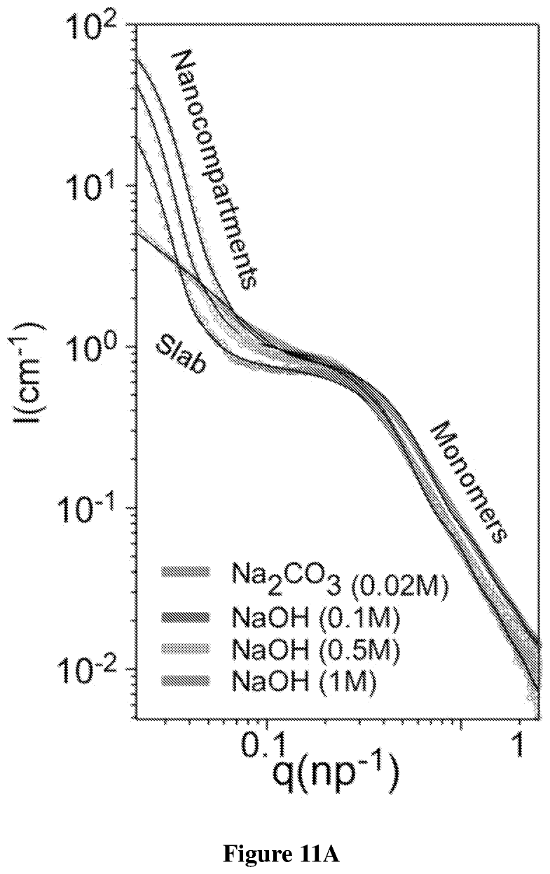

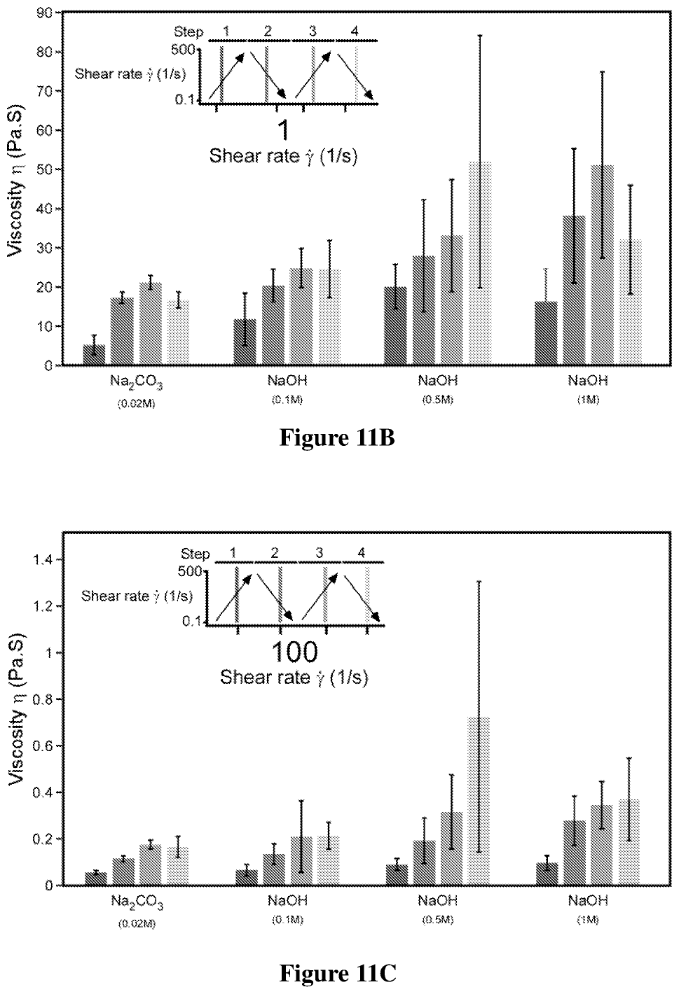

SAXS analysis was performed for soluble silk fibroin on the standard Na₂CO₃-based protocol compared to NaOH-treated samples. The results for Na₂CO₃-based protocol contain a very low amount of nanocompartments and more slab compared to the NaOH-treated samples. Nano-compartments is one way to stabilize soluble fibroin in the silkworm gland. SAXS analysis was performed on native fibroin (taken directly from the silkworm gland), demonstrating a very similar behavior to the NaOH-treated samples (FIG. 11A and Table 4).

FIG. 4A shows a gel electrophoresis analysis of silk fibroin protein molecules. FIG. 11A shows the small-angle x-ray scattering (SAXS) analysis of soluble silk fibroin, showing azimuthally-interacted background-subtracted solution X-ray scattering absolute intensity, I, as a function of the magnitude of the scattering vector, q, from native silk and soluble RSF obtained from the different degummed approaches.

The combination of models that were used to fit the SAXS data are shown in Table 4. The model type (Sphere, Disk, or Slab), the radii of the model, R, the polydispersity of the radii (AR), the heights of the disks or slab, and the mass fractions of the models are shown.

TABLE 4

| | Model | | ±ΔR | Height | Width | Length | Mass |
|---|---|---|---|---|---|---|---|
| Sample | Type | R [nm] | [nm] | [nm] | [nm] | [nm] | Fraction |
| Na₂CO₃ | Sphere | 23 ± 2 | 1.4 | — | — | — | 3.00e−04 |
| (0.02M) | Disk | 3.7 ± 0.2 | 1.2 | 0.63 ± 0.05 | — | — | 9.99e−01 |
| | Slab | — | — | 1 | 90 ± 20 | 250 ± 50 | 1.26e−02 |
| NaOH | Sphere | 55 ± 5 | 5.5 | — | — | — | 6.93e−08 |
| (0.1M) | Disk | 5.5 ± 0.3 | 0 | 0.6 ± 0.1 | — | — | 9.99e−01 |
| NaOH | Sphere | 73 ± 3 | 5.5 | — | — | — | 3.37e−08 |
| (0.5M) | Disk | 5.8 ± 0.2 | 0 | 0.6 ± 0.1 | — | — | 9.99e−01 |
| NaOH | Sphere | 74 ± 3 | 5.5 | — | — | — | 3.30e−09 |
| (1M) | Disk | 5.3 ± 0.2 | 0 | 0.6 ± 0.1 | — | — | 9.99e−01 |

FIGS. 11B and 11C show rheology analysis of changes in viscosity in response to applied shear, with increasing shear rate from 0.1 s⁻¹ to 500 s⁻¹ and back from 500 s⁻¹ to 0.1 s⁻¹ twice. The following samples were analyzed: RSF solution obtained via chemical resolubilization of the silk fibers degummed in the presence of Na₂CO₃, and NaOH 0.5M.the graph demonstrates the viscosity and shear rate of 1 (FIG. 11B) and for 100 (FIG. 11C) with standard deviation (mean±sd, n≥3).

### Example 17

Rheology of Reconstituted from Degummed Silk Fibroin Fibers and its Structural Hierarchy

To show the effect of different degumming procedures on the rheological behavior of reconstituted silk fibroin (RSF) fluid, changes in viscosity in response to the applied shear were measured, with the shear rates increasing from 0.1 s⁻¹ to 500 s⁻¹ and decreasing back from 500 s⁻¹ to 0.1 s⁻¹. Comparison between rheological characteristics of RSF were shown, obtained via different degumming protocols followed by chemical re-solubilization, and native silk fibroin (NSF) extracted directly from B.mori silkgland via dissection. The viscosity values recorded for RSF solutions obtained following the protocol that includes degumming step in the presence of 0.5M and 1M NaOH, was significantly higher than those obtained via standard degumming protocol (FIG. 3C). Such values are comparable with native silk. At the maximum shear rate, (500 s⁻¹), the viscosity for silk solutions reconstituted from fibers degummed with 0.5M and 1M NaOH were 0.03, and 0.05 Pa·s, respectively. However, the viscosity for solutions reconstituted from fibers degummed with 0.1M NaOH at the shear rate 500 s⁻¹ was 0.25 Pa·s. At the minimum shear rate (0.1 s⁻¹), the viscosity for RSF solutions (degumming steps 0.5M and 1M NaOH were 501.17, and 644.53 Pa·s, respectively and the for 0.1M NaOH it was 374 Pa·s. In contrast, RSF that derived from degummed fibers with NaOH 0.1 were higher than Na₂CO₃ (for 500 s⁻¹ shear rate 0.027 Pa·s. and for 0.1 s⁻¹ shear rate the obtained value is 285.5 Pa·s.), but not significantly. This observation can be explained by insufficient concentration of NaOH for effective removal of the gum layer, that further affect the quality of the RSF solution, similarly to Na₂CO₃.

### Example 18

### Dynamic Mechanical Analysis (DMA)

The DMA tests were performed on a TA Q850 under DMA multi-frequency strain mode. The standard polymer test parameters were used: (i) the temperature ramp rate at 3° C./min, (ii) the frequency at 1 Hz, and (iii) stress control of 3×10⁻³ N. Preload force equivalent to 0.012N stress was applied to keep the testing fiber in tension throughout that the dynamic oscillation. Note that temperature scans are only shown with increasing temperature. The DMA procedures were on full-range temperature scans from 27° C. to +270° C. (see FIG. 10A).

### Example 19

### Small-Angle X-Ray Scattering (SAXS) Measurements

Solution SAXS measurements were performed at ID02 beamline at the European Synchrotron Radiation Facility (ESRF), using a beam size of 32.4×145 m² (vertical and horizontal, respectively), photon energy of 12.23 keV, Eiger2 4M (Dectris AG) detector, sample-to-detector distance of 3.114 m, and exposure time of 0.1 s. SAXS models were computed by X+ software, using a water electron density of 333 e-mm⁻³. Data was fitted to a linear combination of uniform disks, and either sphere and/or rod geometries.

Based on the contribution of the intensities of each model at zero scattering vector and the volume of each model, the mass fraction was computed for disks (0.9871), spheres (0.0003), and fibrils (0.0126) in the modeled red curve, presented in FIG. 11A.

### Example 20

### Cross-Sectional and Volumetric Analysis of Degummed Fibroin Fibers—Establishing ‘True’ Values

Prior to evaluation of the mechanical performance of the silk fibers treated with newly developed NaOH-based formulation, there is need in determining the cross-sectional area and fibers' volume—parameters which will be further included into calculations. To measure the fibers' cross-sectional area and to calculate the fibers' volume, the present disclosure combines above mentioned fluorescence assays (intrinsic fluorescence and Nile red staining), with confocal microscopy analysis (FIGS. 1A to 1D) and image processing techniques (FIGS. 1C and 1D, FIG. 11). Thousands of z-stack confocal images were collected (see Table 5) that were further post-processed into a reconstructed 3D model using “Imaris” software (FIGS. 1C and 1D).

TABLE 5

| | | Number of | | Standard | |
|---|---|---|---|---|---|
| Sample | Fluorescence signal | cross-section | Mean (μm²) | Deviation (μm²) | Median (μm²) |
| Untreated | Nile red (635 nm) | 41443 | 86.6 | 15.2 | 88.2 |
| silk-fiber | Intrinsic | 25596 | 206.3 | 38.9 | 199.6 |
| | fluorescence (443 nm) | | | | |
| Na₂CO₃ (0.02M) | Nile red (635 nm) | 11766 | 105.3 | 8 | 106.7 |
| | Intrinsic | 11766 | 103.2 | 8.6 | 103.1 |
| | fluorescence (443 nm) | | | | |
| NaOH (0.1M) | Nile red (635 nm) | 11924 | 100.2 | 7.4 | 98.7 |
| | Intrinsic | 11924 | 95 | 5.4 | 95.7 |
| | fluorescence (443 nm) | | | | |
| NaOH (0.5M) | Nile red (635 nm) | 11762 | 96.3 | 6.7 | 95.4 |
| | Intrinsic | 11762 | 93.2 | 4.7 | 92.7 |
| | fluorescence (443 nm) | | | | |
| NaOH (1M) | Nile red (635 nm) | 11552 | 84 | 12.4 | 82 |
| | Intrinsic | 11552 | 91.8 | 13.5 | 86.5 |
| | fluorescence (443 nm) | | | | |

In order to extrapolate cross-sectional parameters for silk fiber components, the post-processing analysis images were converted into the binary format to calculate the cross-sectional area for each component separately and as combined, using a MatLab script. The obtained values were then used in mechanical calculations, which are described in detail herein. The cross-sectional analysis showed inconsistencies and large STD for silk fibers degummed in presence of Na₂CO₃ (see FIG. 6A and Table 5), while the narrowest STD reported for NaOH 0.5M treatment (FIG. 6c and Table 5). Such differences likely originate from better solubility of the sericin component in NaOH and absence of thermal damage in fibroin protein fibers.

The summary of cooperative analysis for the measured cross-sectional area of untreated fibers, fibers degummed via standard approach and fibroin fibers treated with NaOH-based solution are depicted in FIG. 2A, FIG. 6F and Table 5. The analysis pointed to existing differences in the effectiveness of sericin removal between the standard method and the developed NaOH-based formulation. At high NaOH molar concentration (1M) events of micron-scale bundle decomposition into the separated nanofibrils (see FIG. 7) were observed. Furthermore, the analysis of measured cross-sectional area, which is shown in FIG. 2A, FIG. 6F and Table 5, revealed that NaOH (0.5M)-based degumming, resulted in the smallest standard deviation (STD) values and almost perfect overlap between the values obtained from confocal microscopy analysis.

Those skilled in the art to which this invention pertains will readily appreciate that numerous changes, variations, and modifications can be made without departing from the scope of the presently disclosed subject matter, mutatis mutandis.

1. A method for removing at least part of a gum layer from silk fibers, said method comprising:
incubating said silk fibers in an incubating medium, said incubating medium comprising a hydroxide; wherein said method is carried out at a temperature ranging between 5 to 60 degrees Celsius and wherein said incubating consists of a single incubating step.

2. The method of claim 1 wherein said incubating medium consists of one hydroxide and water.

3. (canceled)

4. The method of claim 1 wherein said hydroxide is selected from: NaOH, LiGH, KOH, RbOH, CsOH, Ca(OH)₂, Sr(OH)₂ or Ba(OH)₂ or combination thereof.

5. The method of claim 1 wherein the concentration of said hydroxide in said incubating medium ranges from between 0.1M to 1M.

6. (canceled)

7. (canceled)

8. The method of claim 1 wherein said incubating is carried out from between 1 minute to 60 minutes.

9. The method of claim 1 further comprising rinsing said silk fibers in a liquid after said incubation.

10. The method of claim 9 wherein the liquid comprises water or a solution.

11. The method of claim 9 further comprising drying said silk fibers after rinsing in said liquid.

12. The method of claim 1 further comprising separating said silk fibers into individual fibers.

13. A degummed silk fiber comprising a core and a gum layer wherein said gum layer comprises 0% to 20% of the cross-sectional area of said degummed silk fiber.

14. The degummed silk fiber of claim 13 wherein said fiber exhibits a Young's modulus of at least 10 GPa, or a Young's modulus which is at least 1.5 greater than for an untreated silk fiber.

15. (canceled)

16. The degummed silk fiber of claim 13 wherein said fiber exhibits a tensile strength of at least 400 MPa, or a tensile strength of at least 1.25 times greater than for an untreated silk fiber.

17. (canceled)

18. The degummed silk fiber of claim 13 wherein said fiber exhibits a strain at break of at least 0.2.

19. The degummed silk fiber of claim 13 wherein said fiber exhibits a Weibull characteristic strength parameter (α) of at least 400 MPa, or a Weibull characteristic strength parameter (α) of at least 1.2 times greater than for an untreated silk fiber.

20. (canceled)

21. The degummed silk fiber of claim 13 wherein said fiber exhibits a Weibull shape parameter (β) of at least 4.

22. A degummed silk fiber of produced by the method of claim 1.

23. A reconstituted silk fibroin material, said material comprising silk fibroin proteins sourced from said degummed silk fiber of claim 13 and a liquid.

24. The reconstituted silk fibroin material of claim 23 wherein said liquid comprises water.

25. The reconstituted silk fibroin material of claim 23 wherein said liquid comprises a salt solution.

26. The reconstituted silk fibroin material of claim 23 wherein said liquid is an aqueous solution, said aqueous solution comprises a salt selected from CaCl₂, KCl, NaCl or any combination thereof.

27. (canceled)

28. (canceled)

29. (canceled)

30. (canceled)

31. (canceled)

32. (canceled)
